(19) United States
(12) Patent Application Publication
(10) Pub. No.: US 2022/0000501 A1
(43) Pub. Date: Jan. 6, 2022

(54) PASSING TENSION MEMBER AROUND TISSUE MASS

(71) Applicant: Empress Medical, Inc., Wilmington, DE (US)

(72) Inventors: Michael Gabriel TAL, Tel Aviv (IL); Gilad MAGNAZI, Hod Hasharon (IL); Ohad HENN, Ramat gan (IL)

(21) Appl. No.: 17/298,873

(22) PCT Filed: Dec. 2, 2019
(86) PCT No.: PCT/US2019/064030

§ 371 Date: Jun. 1, 2021

Related U.S. Application Data
(60) Provisional application No. 62/774,249, filed on Dec. 2, 2018.

(30) Foreign Application Priority Data
Aug. 13, 2019 (US) 16539800

(51) Int. Cl. A61B 17/221

(52) U.S. Cl. CPC A61B 17/221; A61B 2017/22094; A61B 2017/2212

(57) ABSTRACT

Disclosed are apparatus and method for forming passage extending along a plane crossing an organ's volumetric region from an entry point to an opposing exit point at a surface of the organ, and for passing a tension member around the volumetric region by pulling the tension member from the exit point to the entry point through the passage. The apparatus can include a rigid outer tube with a tip for penetrating the organ and reach a penetration depth; an inner needle with elastic body configured to pass straightened through outer tube lumen and to partially protrude and voluntarily flex to a curved form greater than the diameter of the volumetric region; and a tension member passer with a pulling portion for engaging with portion of tension member and for pulling the tension member when withdrawn.

### CROSS-REFERENCE TO RELATED APPLICATIONS

This application claims the benefit under 35 U.S.C. § 119(e) of U.S. Provisional Patent Application No. 62/774,249, filed on Dec. 2, 2018, titled “APPARATUS FOR PASSING TENSION MEMBER AROUND TISSUE MASS AND METHOD OF USE THEREOF”, and U.S. patent application Ser. No. 16/539,800, filed on Aug. 13, 2019, titled “CAUSING ISCHEMIA IN TUMORS”, the entire contents of which are hereby incorporated by reference herein.

### FIELD OF THE INVENTION

The present disclosure, in some embodiments thereof, relates to devices and methods for passing artifacts (e.g., wires or sutures) around target tissues within a body of a subject, and more particularly, but not exclusively, to devices and methods for encompassing a tissue mass (e.g., tumor) with a tension member applicable for causing ischemia and/or necrosis thereto.

### BACKGROUND OF THE INVENTION

A uterine fibroid (also referred to as a “myoma”) is a benign tumor that is fed by the uterine artery and grows within the muscle tissue of the uterus. Myomas are solid fibrous tissue growing as a single nodule or in clusters and may range in size from about 1 mm to more than 20 cm in diameter. Myomas are the most frequently diagnosed tumor in the female pelvis and the most common reason for a woman to undergo hysterectomy. The prevailing symptoms of myomas include heavy menstrual bleeding, prolonged menstrual periods, pelvic pressure or pain and lower urinary tract symptoms (LUTS).

FIG. 1 illustrates an exemplary uterus with three types of fibroids. Uterine Fibroids are classified by their location which effects the symptoms they may cause and how they can be treated. Fibroids that are inside the cavity of the uterus (Submucous fibroids) often cause bleeding between periods and severe cramping. Some submucous fibroids are partially in the cavity and partially in the wall of the uterus. They too can cause heavy menstrual periods (menorrhagia), as well as bleeding between periods and are harder to remove in an hysteroscopic resection. Intramural fibroids are in the wall of the uterus and can range in size from microscopic to larger than a grapefruit. Many intramural fibroids do not cause problems until reaching a certain size. Subserous fibroids are found on the outside wall of the uterus and may even be connected to the uterus by a stalk (pedunculated fibroid). Known devices, systems, and methods for treating uterine fibroids suffer from a variety of limitations and drawbacks.

### SUMMARY OF THE INVENTION

The present disclosure, in some embodiments thereof, relates to devices and methods for passing artifacts (e.g., wires or sutures) around target tissues within a body of a subject, and more particularly, but not exclusively, to devices and methods for encompassing a tissue mass (e.g., tumor) with a tension member applicable for causing ischemia and/or necrosis thereto.

In certain embodiments, there is provided an apparatus for passing a tension member around a volumetric region of an organ. The apparatus may include at least one of:

(a) a rigid outer tube comprising a sharp outer tube tip and an outer tube lumen with an outer tube opening in proximity to the outer tube tip;

(b) an inner needle comprising an elastic needle body curved at least in part thereof, the inner needle ending with a sharp needle tip and enclosing an inner needle lumen with an inner needle opening being in proximity to the needle tip, the inner needle body being configured to pass straightened through the outer tube lumen and to partially protrude via the outer tube opening, such that a protruding portion of the inner needle body is allowed to voluntarily flex to a curved form having diameter equal to or greater than diameter of the volumetric region; and

(c) a tension member passer comprising a tension member passer body, sized for passing through the inner needle lumen, and a tension member pulling portion configured for engaging with a portion of the tension member and for continuously applying a pulling force to the engaged portion of the tension member when the tension member is withdrawn with the tension member passer;

In some embodiments, the apparatus is configured for forming a passage through the organ, the passage extending along a plane crossing the volumetric region from an entry point at a surface of the organ, located in front of a first side of the volumetric region, to an exit point at the surface of the organ, located in front of a second side of the volumetric portion opposite to the first side, and the apparatus is further configured for passing the tension member around the volumetric region by pulling the tension member from the exit point to the entry point through the passage.

In some embodiments, the volumetric region of the organ includes a tissue mass comprising at least a portion of a tumor.

In some embodiments, the outer tube is movable relative to a covering portion of the apparatus until the outer tube tip extends a chosen uncovered length from a distal edge of the covering portion, the distal edge is configured to resist penetration into soft tissue to inhibit insertion of the outer tube to a depth greater than the uncovered length.

In some embodiments, the needle body includes a first segment having a first centerline, and a second segment having a second centerline, the second segment adjoins with a proximal end thereof to a distal end of the first segment and with a distal end thereof to a proximal end of a tip segment, wherein, when the needle body is in an unstressed relaxed form, the first centerline has a first radius of curvature, at least along a portion thereof being adjacent to the first segment distal end, and the second centerline has a second radius of curvature, at least along a portion thereof being adjacent to the second segment proximal end, the second radius of curvature is smaller than the first radius of curvature.

In some embodiments, a ratio between the second radius of curvature and the first radius of curvature is within a range of 1/10 to ⅓.

In some embodiments, when the needle body is in the unstressed relaxed form, the first segment subtends a first subtended angle and/or the second segment subtends a second subtended angle, wherein the second subtended angle is smaller than the first subtended angle.

In some embodiments, the first subtended angle is within a range of 200° to 300°, and/or the second subtended angle is within a range of 10° to 80°.

In some embodiments, the first radius of curvature is within a range of 15 mm to 45 mm when the needle body is in the unstressed relaxed state.

In some embodiments, the elastic needle body is configured with elastic resistance to straightening within a range of 2 N to 20 N.

In some embodiments, the apparatus is configured such that the protruding portion exits the outer tube opening with a needle exit angle δ within a range of 10° to 80°, relative to the outer tube.

In some embodiments, the tension member passer body is flexible and elastic.

In some embodiments, the tension member pulling portion includes a securing member forming a loop with the tension member passer body.

In some embodiments, the tension member passer body has a curved or bent portion forming a deviated distal end portion inclined relative to remainder of the tension member passer body.

In some embodiments, the deviated tension member passer distal end portion forms with rest of the tension member passer body a deviation angle within a range of 15° to 55°.

In some embodiments, the tension member pulling portion includes a securing wire portion extending from a first location on the tension member passer body, distally to the curved or bent portion, to a second location on the tension member passer body, proximally to the curved or bent portion.

In some embodiments, the securing wire portion is similar in length to length of a segment of the tension member passer body extending from the first location to the second location.

In some embodiments, the securing wire portion is configured to undergo increased tension when the deviated tension member passer distal end portion is forced to align with rest of the tension member passer body.

In some embodiments, the deviated tension member passer distal end originates at the first location and extends in a straight form at least 10 mm in length.

In some embodiments, the curved or bent portion of the tension member passer body is configured with elastic resistance to straightening within a range of 0.1 N to 1 N.

In some embodiments, the apparatus further comprising a console, optionally formed as a handheld device.

In some embodiments, the apparatus further comprising an inner needle protrusion controller configured to operatively control advancement of the inner needle within the outer tube.

In some embodiments, the apparatus further comprising a tension member passer protrusion controller configured to operatively control advancement of the tension member passer body within the inner needle.

In some embodiments, the apparatus further comprising a forceps head fixedly positioned distally to the console and activatable via the console, configured for selective grasping of the organ adjacent to the entry point and/or the exit point, for holding the grasped organ at a fixed distance relative to the console.

In some embodiments, the forceps head is configured in a form of tenaculum having two hinged tenaculum arms, each tenaculum arm includes a slender sharp-pointed hook configured to penetrate through the organ surface into the organ when the forceps head is operated to grasp the organ surface.

In some embodiments, the outer tube is slidable distally relative to the forceps head such that the outer tube tip is extendable distally beyond the forceps head, wherein the apparatus is configured to prevent extension of the outer tube tip distally beyond the forceps head over a predetermined maximal penetration depth.

In certain embodiments, there is provided a method for passing a tension member around a volumetric region of an organ. The method may include at least one of the following steps (not necessarily in the listed order):
using a rigid outer tube, comprising a sharp outer tube tip and an outer tube lumen with an outer tube opening in proximity to the outer tube tip, penetrating into the organ such that the outer tube tip reaches a penetration depth;
passing an inner needle in the outer tube lumen, the inner needle includes an elastic needle body curved at least in part thereof, ending with a sharp needle tip and enclosing an inner needle lumen with an inner needle opening in proximity to the needle tip;
piercing a curved passage with the needle tip around the volumetric region with a protrusion length of a protruding portion of the inner needle body, by pushing the inner needle via the outer tube opening and allowing the protruding portion to voluntarily flex to a curved form having diameter equal to or greater than diameter of the volumetric region;
advancing a tension member passer comprising a tension member passer body and a tension member pulling portion, in the inner needle lumen and via the inner needle opening, until the tension member pulling portion exits the organ at an exit point opposing the entry point relative to the volumetric region; and
drawing the tension member into and through the curved passage by pulling the tension member passer with the secured tension member.

In some embodiments, the drawing includes extending the tension member around the volumetric region such that one end of the tension member projects from the entry point and another end of the tension member projects from the exit point.

In some embodiments, the organ is an internal organ located within a body of a live subject, and the method further comprising forming a surgical route from outside the body of the subject and delivering the outer tube through the surgical route until the outer tube tip reaches the organ.

In some embodiments, the organ is a uterus.

In some embodiments, the volumetric region of the organ includes a tissue mass comprising at least a portion of a tumor.

In some embodiments, the method comprising ending the piercing with positioning the needle tip at a chosen distance from the surface of the organ, so as to form a needle tip angle between the needle tip and the surface of the internal body region.

In some embodiments, the distance is smaller than 3 cm, and/or the needle tip angle is within a range of 10° to 60°.

In some embodiments, the defining includes defining a penetration angle between the outer tube and a perpendicular line to the surface of the internal body region at the entry point, wherein the protrusion length subtends a subtended angle is at least 270° minus the penetration angle.

In some embodiments, the penetrating, the passing, the piercing, the advancing and/or the securing is repeated, each repetition is performed using a different implanted tension member, a different entry point and a different exit point.

In some embodiments, the method comprising:
using a forceps head, grasping the organ adjacent to the entry point and/or the exit point for holding the grasped organ before the penetrating with the outer tube.

In some embodiments, the outer tube is slidably connected to a console and the forceps head is fixedly positioned distally to the console, wherein the outer tube is slidable distally relative to the forceps head such that the outer tube tip is extendable distally beyond the forceps head up to a predetermined maximal penetration depth.

All technical or/and scientific words, terms, or/and phrases, used herein have the same or similar meaning as commonly understood by one of ordinary skill in the art to which the invention pertains, unless otherwise specifically defined or stated herein. Exemplary embodiments of methods (steps, procedures), apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict, the patent specification, including definitions, will control.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative description of some embodiments of some embodiments. In this regard, the description taken together with the accompanying drawings make apparent to those skilled in the art how some embodiments may be practiced.

In the drawings:

FIG. 1 schematically illustrates a frontal cross-sectional view of an exemplary female uterus having different types of fibroids growing therein;

FIGS. 2A-2B schematically illustrate a cross-sectional view of an illustrative tumor being radially compressed using one or more tension members, either about entire volume of the tumor (FIG. 2A) or about a number of volumetric portions of the tumor (FIG. 2B), according to some embodiments;

FIGS. 3A-3D schematically illustrate views of different exemplary fibroids treated using one or more tension members, according to some embodiments;

FIGS. 4A-4C schematically illustrate side cross-sectional views of components of an exemplary apparatus for passing a tension member around a tissue mass, according to some embodiments;

FIGS. 5A-5D schematically illustrate exemplary scenarios representing steps in an exemplary method for using the exemplary apparatus referred to in FIGS. 4A-4C, according to some embodiments;

FIGS. 6A-6H schematically illustrate exemplary scenarios representing steps in an exemplary method for passing a tension member around a tissue mass within an internal body region, using the exemplary apparatus referred to in FIGS. 4A-4C, according to some embodiments;

FIGS. 7A-7B illustrate respectively an isometric view and a partial side cross-sectional view of an exemplary apparatus for passing a tension member around a tissue mass, according to some embodiments;

FIGS. 8A-8B illustrate respectively a full isometric view and a zoom-in partial isometric view of an exemplary outer tube uncovering mechanism provided in the exemplary apparatus shown in FIG. 7A, according to some embodiments;

FIGS. 9A-9B illustrate respectively a full isometric view and a zoom-in partial isometric view of an inner needle advancing mechanism provided in the exemplary apparatus shown in FIG. 7A, according to some embodiments;

FIGS. 10A-10B illustrate respectively a full isometric view and a zoom-in partial isometric view of a tension member passer advancing mechanism provided in the exemplary apparatus shown in FIG. 7A, according to some embodiments;

FIGS. 11A-11B illustrate respectively a full side view and a zoom-in partial side view of an exemplary inner needle in an unstressed relaxed state, according to some embodiments;

FIG. 12 illustrates a side view of an exemplary tension member passer in an unstressed relaxed state, according to some embodiments;

FIGS. 13A-13D illustrate views of the apparatus shown in FIG. 7A representing several exemplary scenarios of operation thereof, according to some embodiments;

FIGS. 14A-14D illustrate isometric and side cross-sectional views of an exemplary apparatus for passing a tension member around a tissue mass, which further comprises a forceps head, according to some embodiments;

FIGS. 15A-15E illustrate views of a distal portion of the apparatus shown in FIG. 14A representing several exemplary scenarios of operation thereof, according to some embodiments;

FIG. 16 illustrates a side view of an exemplary surgical needle applicable as an inner needle of the apparatus shown in FIG. 14A, according to some embodiments; and

FIGS. 17A-17D illustrate a distal portion of the apparatus shown in FIG. 14A representing several exemplary scenarios of advancing the needle shown in FIG. 16, according to some embodiments.

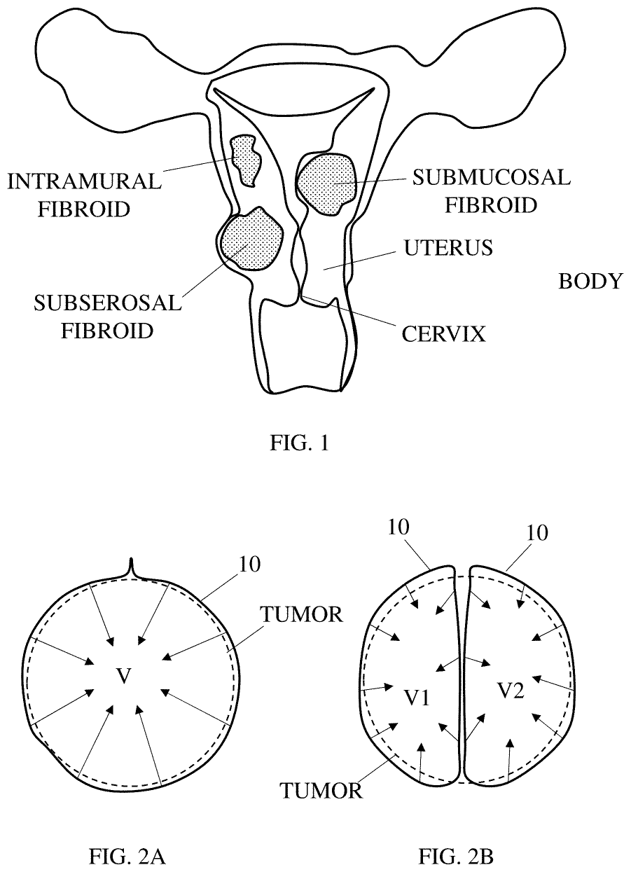

### DETAILED DESCRIPTION

Certain embodiments relate to devices and methods for passing artifacts (e.g., wires or sutures) around target tissues within a body of a subject, and more particularly, but not exclusively, to devices and methods for surrounding or encompassing a tissue mass (e.g., tumor) with a tension member applicable for causing ischemia and/or necrosis thereto. One or more tension members are applied, according to methods described herein, around or through a target tumor, and are put under tensioning force in a manner that triggers, supports and/or induces tumor suppression.

A “tension member”, as referred to in current disclosure, relates to any flexible slender member that can withstand tension forces of at least 0.1 Kg, optionally at least 0.5 Kg, optionally at least 1 Kg, without failure (e.g., plastic deformation, tear, or breaking). In some embodiments in this disclosure, tension members cannot withstand significant compression and/or lateral forces without, breaking, collapsing or altering shape. Exemplary tension members may include medical or surgical grade wires, filaments or cables, such as sutures (e.g., biodegradable sutures) and cable ties.

In embodiments, tension members are deployed and directly affect (cause) a continuous pressure within the tumor (interstitial pressure), optionally above 22.5 mmHg, thus inhibiting blood flow into the tumor. Optionally, additionally or alternatively, tension members are deployed to path over blood vessels nourishing the tumor and are configured and taut sufficiently so as to impinge the blood vessels and block blood flowing therethrough. Blocking blood supply to the tumor for several hours leads to fibroid ischemia and eventually to necrosis of the tumor cells.

As described, the tension members can be placed around entire volume of the tumor (fibroid), optionally including portions of other tissues surrounding it, or around one or more smaller volumetric portions thereof. It may be advantageous to prefer the first option of surrounding the entire tumor (fibroid) and/or avoid passing a tension member across tumor volume especially due to sharp increase in density when entering the fibroid or the possibility the tumor is cancerous, so that puncturing therethrough increases risk of cancer spreading to surrounding tissues and blood system. Nevertheless, in some procedures it may be found advantageous to pass a tension member through the tumor such as in anatomies imposing difficulties to fully encompass the tumor.

FIGS. 2A-2B schematically illustrate a cross-sectional view of an exemplary target tissue mass in a form of a tumor being compressed using one or more tension members 10, either about entire volume V of the tumor (FIG. 2A) or about a number of volumetric portions V1 and V2 of the tumor (FIG. 2B). Optionally, a plurality of tension members 10 are deployed, each one encompasses more than half a circumference of the tumor or the volumetric portion, optionally more than two thirds the circumference, optionally close to a full the circumference. When under a chosen tensioning force (e.g., predetermined, measured and/or calculated), tension members 10 can be applied to affect radial compression of the surrounded volumetric portion. A plurality of tension members can be arranged around a volumetric portion of the tumor, such that the combined effect of all tension members thereto is compression towards the volumetric center of the tumor or of the volumetric portion. Tension members 10 are passed spaced apart with each other relative to a center of the tumor or the volumetric portion, optionally evenly spaced apart.

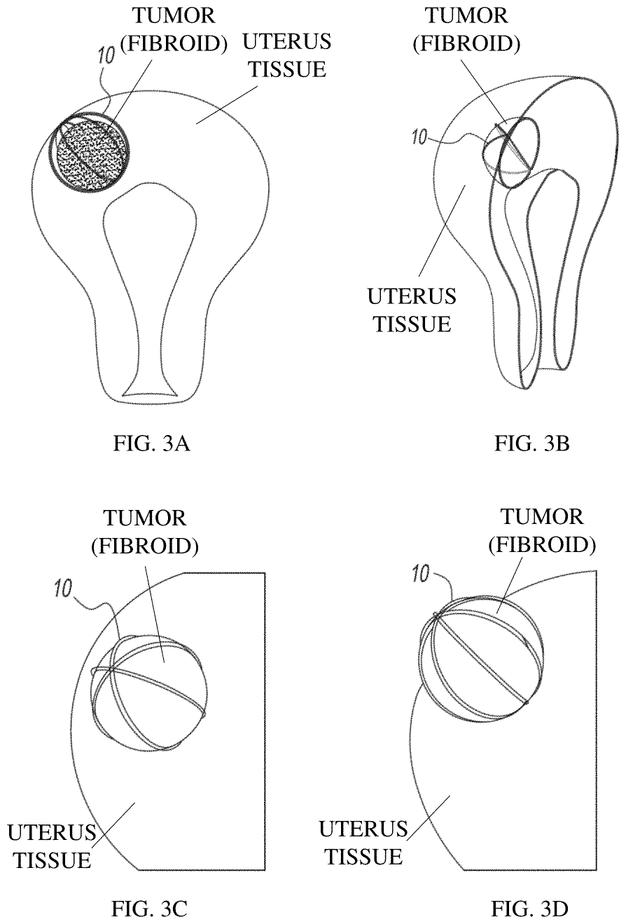

FIGS. 3A-3D schematically illustrates views of different exemplary configurations of fibroids following treatment, according to some embodiments. In some embodiments, as shown in FIG. 3A, one or more tension members 10 may be provided (implanted) around the fibroid when passing partially or fully through a (healthy) uterus tissue surrounding the fibroid, particularly in cases of intramural fibroids. Optionally, additionally or alternatively, one or more tension members 10 may be provided through the fibroid tissue, such as through its center or in proximity thereto, as shown in FIG. 3B. The number of tension members used can be determined according to need or tumor size or type, for example two tension members, three tension members (FIG. 3C), four tension members (FIG. 3D), five tension members, six tension members, eight tension members, or more. In some embodiments, one or more tension members 10 can be secured to the tumor and/or surrounding tissue by way of suturing or tying around the tumor (as shown in FIGS. 2A and 3A, for example) or a volumetric portion thereof (as shown in FIG. 2B, for example).

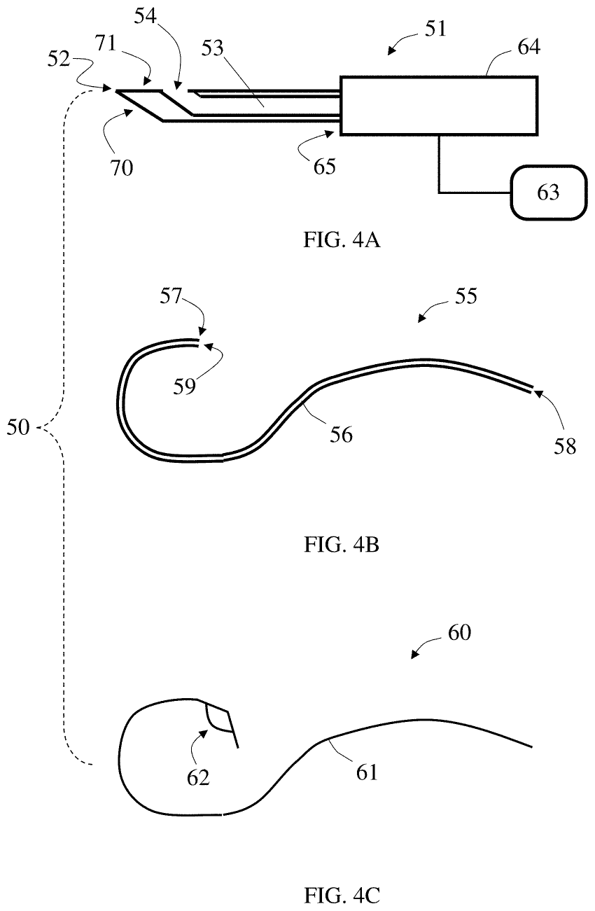

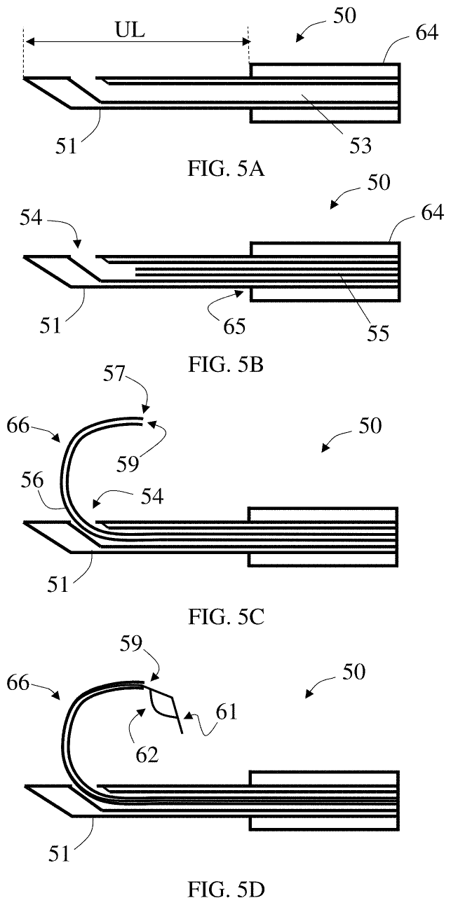

FIGS. 4A-4C schematically illustrate side cross-sectional views of components of an exemplary apparatus 50 for passing a tension member around a tissue mass. FIGS. 5A-5D schematically illustrate exemplary scenarios representing steps in an exemplary method for using apparatus 50. FIG. 4A shows a rigid outer tube 51 which comprises a sharp outer tube tip 52 and an outer tube lumen 53 opened to an outer tube opening 54 formed as a lateral opening in proximity to outer tube tip 52. Outer tube 51 includes a bevel configured with a bevel face 70 opposing a straight side 71 that encloses outer tube side opening 54. An outer tube uncovering mechanism 63 is provided with outer tube 51 and configured for fixating a chosen uncovered length UL of outer tube 51 relative to a tube cover 64 covering remaining length of outer tube 51 (as shown in FIG. 5A). Tube cover 64 has a distal edge 65 (e.g., outer diameter), being wider substantially from boundary (e.g., outer diameter) of outer tube 51, configured to resist penetration of outer tube 51 into soft tissue beyond a depth penetrable with uncovered length UL.

FIG. 4B shows an inner needle 55, in an unstressed relaxed length (in which no external forces or internal stresses are applied in a manner sufficient to deform its size and/or shape, at least not significantly and/or visually), which comprises an elastic needle body 56 ending with a sharp needle tip 57 and enclosing an inner needle lumen 58. Inner needle lumen 58 is opened to an inner needle opening 59 in proximity to needle tip 57. Inner needle 55 is configured to pass straightened through outer tube lumen 53 due to its flexibility and the constraining rigid boundaries of outer tube lumen 53 affected by surrounding wall of outer tube 51 (As shown in FIG. 5B). Once inner needle 51 partially protrudes via outer tube opening 54, the protruding portion 66 of inner needle 51 can voluntarily flex (by its elasticity properties) to a curved form, as shown in FIG. 5C. When in its curved form, inner needle 55 is configured to pierce a curved passage around a target tissue mass, by rotationally advancing through the soft tissue surrounding the tissue mass, when pushed via outer tube opening 54.

Since that apparatus 50 is configured to pass tension members around a tissue mass such as fibroids, which can be of different sizes, shapes and/or depth (relative to surface of an internal body organ, for example), it may be advantageous in some scenarios to preset a penetration length, from within a range of allowed selectively fixable lengths, which is deriving from, and equal to, the uncovered length UL. This measured penetration of outer tube 51 will allow outer tube opening 54 to be positioned near the outer periphery of the target tissue mass, such that the protruding portion 66 of inner needle 55 can be curved beyond and around the distal boundaries of the tissue mass in proximity thereto. In some embodiments, uncovered length UL is determined in accordance with positioning outer tube opening 54 in proximity to a chosen part of the tissue mass, for example near its middle. Predetermining uncovered length UL may be performed in advance using analysis of invasive or noninvasive imagery.

FIG. 4C shows a tension member passer 60, in an unstressed relaxed state, which comprises a tension member passer body 61, sized for passing through inner needle lumen 58, and a tension member passer securing member 62, optionally a wire forming a snare-like structure with tension member passer body 61, configured for securing a portion of a tension member to the tension member passer body 61. As shown in FIG. 5D, tension member passer 60 is advanced through inner needle lumen 58 until tension member passer securing member 62 protrudes (fully or partially) from inner needle opening 59. As will be described in details below, a tension member can be secured to tension member passer body 61 with tension member passer securing member 62, then the tension member can be withdrawn towards and/or into outer tube lumen 53 (via outer tube opening 54) by pulling it with tension member passer 60, optionally together with inner needle 55.

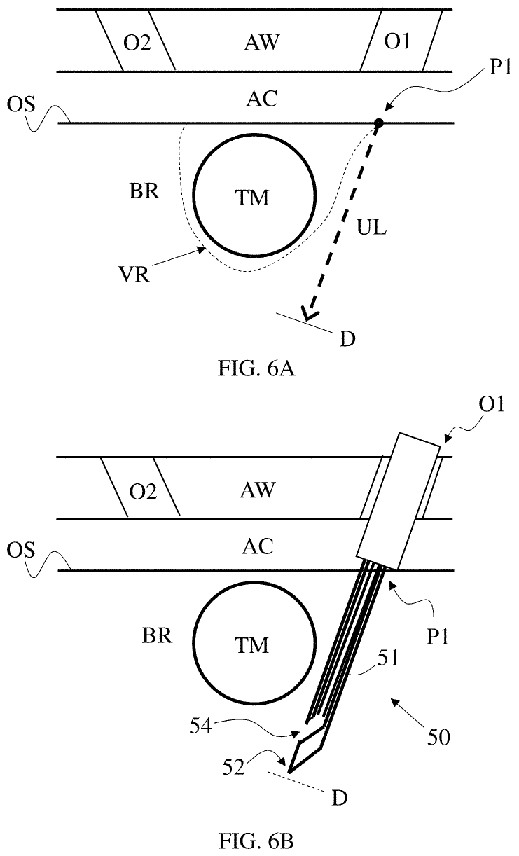

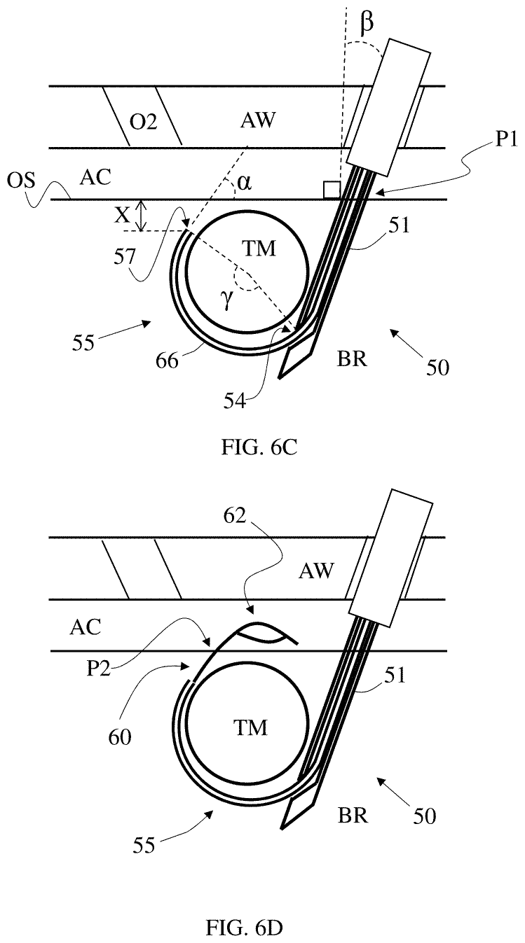

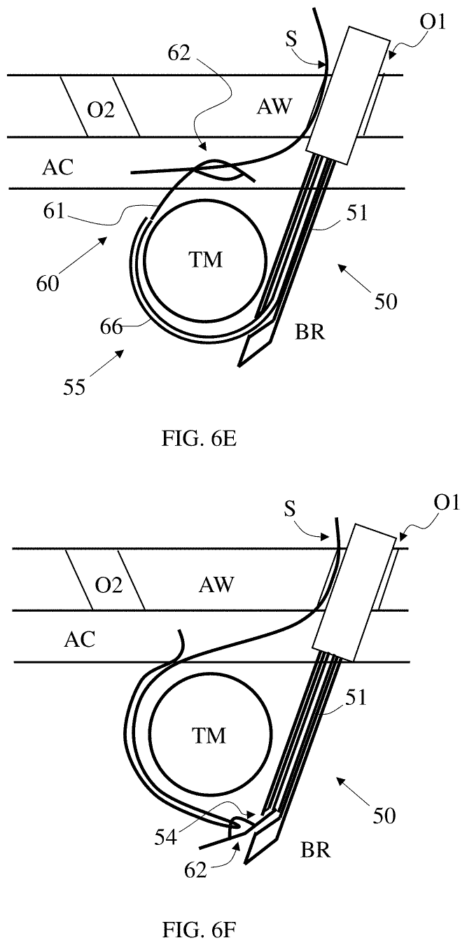

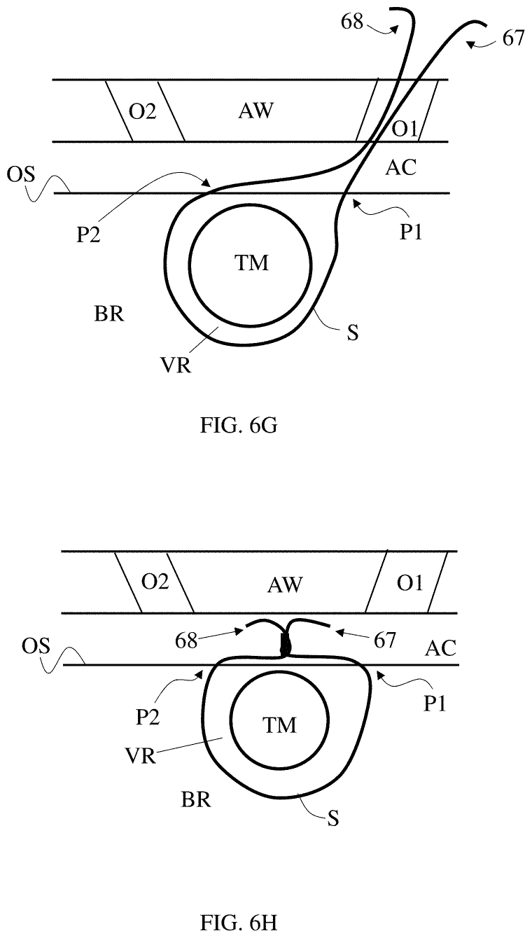

FIGS. 6A-6H schematically illustrate exemplary scenarios representing optional steps in an illustrative method for passing a tension member S around a volumetric region VR of an organ. Volumetric region VR includes a tissue mass TM optionally comprising at least a portion of a tumor. The organ, optionally a uterus of a female subject, is illustrated and referred to in part thereof as a body region BR. In various embodiments, the method is intended for facilitating selective volumetric compression of volumetric region VR and/or tissue mass TM to increase pressure (optionally an interstitial pressure) within the volumetric region VR above a threshold level that is sufficient to cause ischemia of tissue mass TM or a tumor therein; and maintaining the pressure above the threshold level for a period sufficient to permit at least a portion of the volumetric region VR to necrotize due to the ischemia.

The volumetric region VR is optionally circumscribed with at least one device of foreign origin relative to the patient, for example a tension member such as a surgical wire, wherein the volumetrically compressing the volumetric region VR and maintaining the elevated internal pressure are achieved via the at least one device. The treated tissue mass TM is optionally a uterine fibroid, and may be one of intramural, subserous or submucosal with respect to the organ it resides in. Optionally, at least a portion of the tissue mass TM is situated intramurally within the organ, and wherein passing the tension member within the organ comprises passing the tension member through an intramural portion of the organ. Passing the tension member through the intramural portion of the organ may comprise passing the tension member around the at least a portion of the tissue mass TM that is situated intramurally within the organ. In some such scenarios, passing the tension member within the organ may comprise passing the tension member exclusively through the intramural portion of the organ and/or the tissue mass TM between the entry point/opening and the exit point/opening.

In order to reach the surface of the organ and treat the tissue mass TM, a surgical access to the organ may be first created from outside the body, which may be formed using minimally invasive techniques or by way of open surgery, for example. At least one of the basic method steps can be performed via the surgical access. The entry point to the organ can be located at a first location on or adjacent to the tissue mass and the exit point can be located at a second location on or adjacent to the tissue mass spaced from the first location, such that the tissue mass TM is located between the entry and exit openings.

Prior to passing the tension member, a passage can be formed around the volumetric region VR and tissue mass TM between the entry and exit points, optionally also forming the entry and exit points (openings), such that the passing can be performed mostly or entirely within the passage, optionally by way of pulling the tension member via the exit point towards the entry point. The passage may be formed using apparatus 50 or any other applicable apparatus or mechanism. For example, an outer tube can be used to create the entry point and positioned through the entry point (opening) into the organ, in proximity to the tissue mass. A curved needle can then be advanced through a lumen of the outer tube around the volumetric region VR.

The volumetric region may be predetermined by a user (practitioner, physician, surgeon, etc.) and passing the tension member may be performed in close fit to and around the tissue mass. Determining the volumetric region may include determining entry and exit points to and from the organ in relation to the tissue mass, and possibly also a particular plane crossing the volumetric region VR and/or tissue mass TM. Passing the tension member may be along a predetermined passage line between the entry point and the exit point. The passage is configured to extend along a plane crossing the volumetric region from an entry point at a surface of the organ, located in front of a first side of the volumetric region, to an exit point at the surface of the organ, located in front of a second side of the volumetric portion opposite to the first side of the organ. The passage line optionally projects across one or more blood vessels feeding the tumor, such that the tightening of the tension member directly causes occlusion of the blood vessels, such as previously discussed.

Passing the tension member may include encompassing more than half a circumference of the tumor with the tension member, and/or it may include winding the tension member or a plurality of additional tension members along separate paths and/or planes around the volumetric region. In case of an additional volumetric region encompasses at least another portion of the tumor, passing the tension member may also include deploying a plurality of windings around the additional volumetric region. The volumetric region optionally encompasses most of a volume of the tissue mass, or its entirety.

The tension member optionally comprises a flexible strip or a wire, such as a suture wire, and may be formed of at least one of implant-grade metal alloy, implant-grade polymer, implant-grade textile, and biodegradable material. In certain embodiments, the tension member is configured with a yield strength or a maximal tension force of at least 25 N (newtons) in order not to prevent failing during tumor compression. Optionally the tension member is configured to yield above about 80 newton or about 100 N (newton) before it can cause cutting in organ tissues resulting from tumor compression by the tension member. Optionally, the tension member is formed as a biodegradable suture wire and is configured to yield under tensioning forces below 25 N (newtons) after the tumor tissues are ischemic or necrotic, for example after a few weeks or months.

With further reference to FIGS. 6A-6H demonstrate various stages of an illustrative method of passing a tension member S, configured as wire (e.g., suture), around a tissue mass TM (e.g., tumor) within an organ or a body region BR. FIG. 6A illustrates an exemplary scenario in which one or more surgical (e.g., minimally invasive or laparoscopic) openings, O1 and O2, are formed to an abdominal wall AW for creating separate surgical passages into an abdominal cavity AC and therethrough to outer surface OS of internal body region BR. Either one of openings O1 and O2 may be a transcutaneous cut or a fixed passage maintained by an artifact such as a trocar or a cannula.

In an optional preliminary step, the user (surgeon, practitioner, etc.) may determine a desired orientation for a tension member to pass within body region BR with respect to tissue mass TM. Such a calculated, selected, and/or predetermined orientation may be spatial or two-dimensional. The user may determine an at least one volumetric region VR which encompasses at least a portion of tissue mass TM. Optionally, alternatively or additionally, the user determines a plane crossing or passing through tissue mass TM on which points of entry and exit to and from the body region BR will be made. Optionally, a penetration depth D is defined, taken from an entry point P1 at surface OS relative to boundaries of tissue mass TM.

A suture passing mechanism, optionally part of apparatus 50, is then put into use. In some instances, a chosen uncovered length UL of outer tube 51 is first set or fixated, which uncovered length UL can be substantially equal to penetration depth D, by adequately withdrawing tube cover 409 (as described above). Alternatively, uncovered length UL is fixed and predetermined. Apparatus 50 is then passed via first laparoscopic opening O1 and then pressed with sharp outer tube tip 52 at a chosen direction against surface OS until penetrating the soft tissue of the body region BR in proximity to tissue mass TM (FIG. 6B). By doing so, tip 52 forms entry point P1 and a first segment of a surgical passage within body region BR around tissue mass TM and/or volumetric portion VR. Apparatus 50 is pushed distally until outer tube tip 52 reaches the predefined penetration depth D, or possibly slightly beyond it, or until outer tube opening 54 is positioned a chosen distance (e.g., a chosen proximal distance) from a distal boundary of the tissue mass TM. For example, the opening 54 may, in some instances, desirably be positioned at a depth that corresponds to about the middle of tissue mass TM (i.e., a distance substantially equal to the radius of tissue mass TM, as spaced from a distal boundary of the tumor). In some embodiments, apparatus 50, or particularly outer tube 51 and/or inner needle 55, is of a chosen size out of a variety of sizes, such that the length between outer tube opening 54 and outer tube tip 52 is about the size of tissue mass TM radius. In such embodiments, penetration depth D will be determined so that if outer tube tip 52 is in proximity to distal boundary of tissue mass TM then outer tube opening 54 is also in proximity to middle of tissue mass TM.

Stated otherwise, in some instances, a plurality of inner needles 55 may be provided. Each needle 55 may have has a pre-curved region with a length and/or radius of curvature that differs from the lengths and/or radii of curvature of the remaining options. A user may choose one inner needle 55 out of the available plurality that will form a passageway of a desired shape, size, and/or orientation around the tumor. In some instances, the outer needle 55 is provided separately from one or more of the inner needles 55. In other instances, the outer needle or tube 51 and a plurality of inner needles 55 are provided together (e.g., are provided in a unitary kit).

As shown in FIG. 6C, inner needle 55 can then be passed in outer tube lumen 58 in a travel length sufficiently for extending protrusion portion 66 in a chosen protrusion length via outer tube opening 54. Accordingly, by pushing inner needle 55 via outer tube opening 54, the surgical passage made in body region BR is extended with a curved segment pierced with needle tip 57 around volumetric region VR and/or tissue mass TM, along span of protrusion portion 66. As described above, protruding portion 66 naturally flexes from a straightened form to regain a preformed curved form; and the curved portion can advance along a curved path through soft tissue surrounding the tissue mass TM so as to facilitate formation of (e.g., via piercing through tissue) the curved passage segment.

Inner needle 55 protrudes from outer tube opening 54 at predetermined distance proximally to penetration depth D (e.g., equal to about the size of tissue mass TM radius). Therefore, since outer tube opening 54 is configured as lateral opening, soft tissue penetrated with outer tube 51 is prevented from entering outer tube lumen 53; the inclined exit of inner needle 55 immediately at boundary of outer tube opening 54 increases the initial piercing power of inner needle 55 into soft tissue surrounding outer tube opening 54, relative to tangential exit; and the portion of outer tube 51 between outer tube opening 54 and outer tube tip 52 increases resistance of outer tube 51 to motions in reaction to inner needle 55 engagement with soft tissue laterally thereto.

Once the penetration depth D is determined, and optionally after outer tube 51 is accordingly positioned in internal body region BR along tissue mass TM, the chosen positioning of inner needle tip 57 and protrusion length of inner needle protruding portion 66 can be determined. In some embodiments, inner needle protruding portion 66 is required to surround a chosen portion of tissue mass TM perimeter (measured in an angle γ subtended by inner needle protruding portion 66), and needle tip 57 is required to be positioned at a chosen distance X from internal body region surface OS and/or at a chosen needle tip angle α formed between tangent projection of inner needle 55 at needle tip 57 and internal body region surface OS. In some embodiments, all dimensions are configured relative to largest cross section of tissue mass TM in a certain direction.

In some embodiments, needle tip angle α is equal to or smaller than 90°, optionally taken within a range of about 10° to about 60°, optionally about 30° to about 45°, so that further penetration by tension member passer 60 until emerging into abdominal cavity AC with tension member passer securing member 62 (as shown in FIG. 6D) will be sufficiently close (e.g., within distance of about 5 cm or less) to entry point P1, yet without risking further curving of tension member passer 60 within internal body region BR and emerging back into abdominal cavity AC. Similarly, distance X is optionally smaller than 5 cm, optionally taken within a range of 0.5 cm to 3 cm, optionally 0.5 cm to 1.5 cm.

Subtended angle γ of inner needle protruding portion 66 is determined according to the target positioning of needle tip 57 relative to entry point P1 and tissue mass TM, as described, and it is also dependent on the magnitude of outer tube 51 penetration angle β (measured relative to perpendicular line to internal body region surface OS at entry point P1). Optionally, Subtended angle γ is greater than 180°−β, optionally particularly at least 225°−β, optionally particularly at least 270°−β.

After formation of the curved portion of the path via the inner needle 55, the tension member passer 60, which may optionally be pre-loaded within inner needle 55, is advanced through inner needle lumen 58 and out of inner needle opening 59 until securing member 62 portion exits internal body region BR at an exit point P2, which can be spaced from (e.g., opposingly located relative to) entry point P1, relative to tissue mass TM (FIG. 6D). Stated otherwise, the entry and exit points P1, P2 may be at opposing sides of the tumor along a surface of the organ (e.g., uterus). As described above, location of exit point P2 can be predetermined or at least selected or determined in advance in correlation with distance and orientation of inner needle tip 57 relative to internal body region outer surface OS. In some instances, it can be desirable for the exit point P2 to be within a range of 2 cm to 5 cm from entry point P1 in order to: keep both points P1, P2 within the user's visual range (e.g., using an endoscope or camera positioned in abdominal cavity AC via a separate channel or surgical opening); in some instances, sufficiently distant from adjacent organs which can be harmed if unintentionally penetrated; and/or effectively tension both ends of tension member (suture) S around tissue mass TM which ends of the tension member S will ultimately emerge from points P1 and P2, as shown in FIG. 6H, for example.

in certain embodiments, tension member S is inserted into abdominal cavity AC through first or second surgical opening O1, O2 (in this example, first opening O1, alongside, through or with apparatus 50) as shown in FIG. 6E. A portion of tension member S is then passed through the lumen of securing member 62, optionally using a surgical tool such as surgical grasper which may be operated via second opening O2, or via first opening O1, or provided with or via apparatus 50. The tension member S therefore can be coupled or secured to tension member passer body 61 and drawn towards and/or into inner needle lumen 58 and/or outer tube lumen 53 by pulling tension member passer 60 with tension member S secured thereto (FIG. 6F). In some instances, a grip of the securing member 62 on the tension member S can increase or be enhanced as the securing member 62 is drawn into the lumen of the tube 51 and/or the inner needle 55, as a loop formed thereby may be resiliently compressed when passing into or through the lumens thereof.

Apparatus 50 is then pulled out from inner body region BR while drawing the captured tension member S, and then removed from patient's body. As a result, tension member S can be left extended around volumetric region and/or tissue mass TM such that one portion or end 67 of tension member S extends from entry point P1 through abdominal cavity AC and out of patient's body, and another portion or end 68 of tension member S extends from exit point P2 through abdominal cavity AC and out of patient's body (FIG. 6G). The two portions 67 and 68 of tension member S can be further manipulated from outside the body, such as for tightening (and/or tumor compressing) and securing (and/or maintaining tumor compression) of tension member S, as shown in FIG. 6H, for example, either by connecting together its two ends 67 and 68 (e.g., by way of tying) and/or by connecting them using an additional component or material (e.g., by way of crimping). In some embodiments, the tension member S loop is fastened while taut thereby affecting continuous compression to target tissue TM. Residual length of tension member S can be trimmed and removed as needed. Tension member S can be made of biodegradable material and left implanted indefinitely.

Some or all steps can repeated, each repetition performed using a different implanted suture, a different entry point and a different exit point.

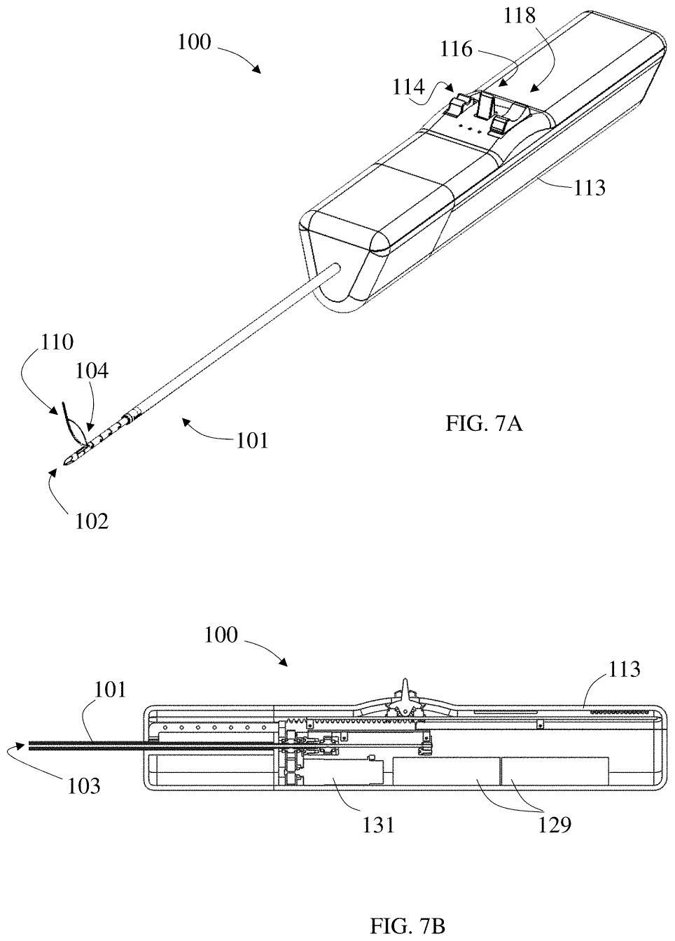

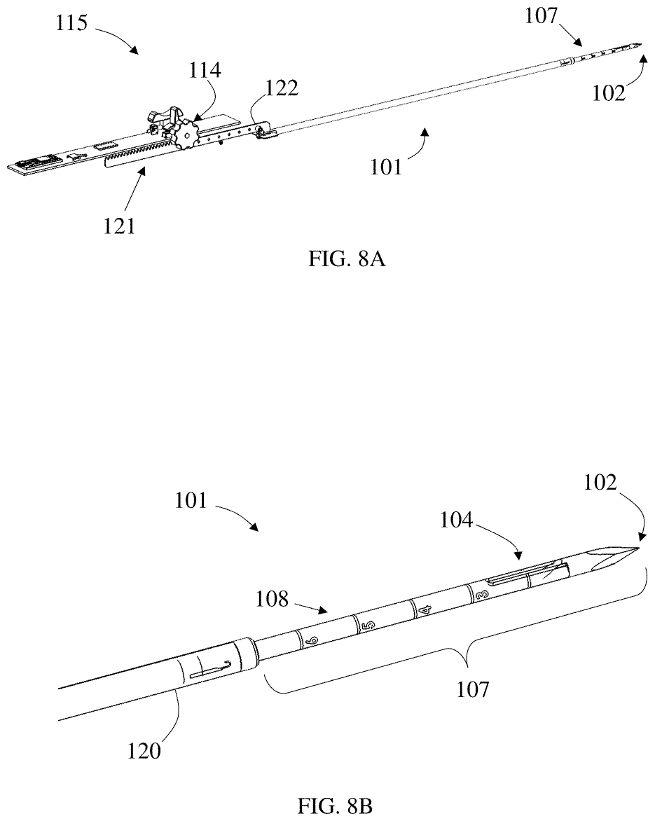

FIGS. 7A-7B illustrate respectively an isometric view and a partial side cross-sectional view of an exemplary apparatus 100 for passing a tension member around a tissue mass. Apparatus 100 is optionally an exemplary implementation or variation of apparatus 50 described above, and it may include some or all embodiments and features of apparatus 50. Apparatus 100 includes a rigid outer tube 101 (shown in greater detail in FIGS. 8A and 8B) comprising a sharp outer tube tip 102 and an outer tube lumen 103 opened to an outer tube opening 104 in proximity to outer tube tip 102. Outer tube 101 is straight and formed of stainless steel, and has a total length within 20 cm to 40 cm. Outer tube lumen 103 has dimeter within range of 3 mm to 12 mm, optionally about 5 mm. Outer tube opening 104 is provided at the side of outer tube 101 (i.e., lateral opening), it is oval in shape with distal portion thereof optionally adjoining with outer tube lumen via a slope, so that inner needle can slide its way to protrude therethrough. Outer tube opening 104 is located at a distance from outer tube tip 102 being about the size of a target tissue mass radius, therefore different outer tube sizes can be chosen according to different tissue masses sizes, or that the distance between outer tube opening 104 and outer tube tip 102 can be selectively fixated accordingly.

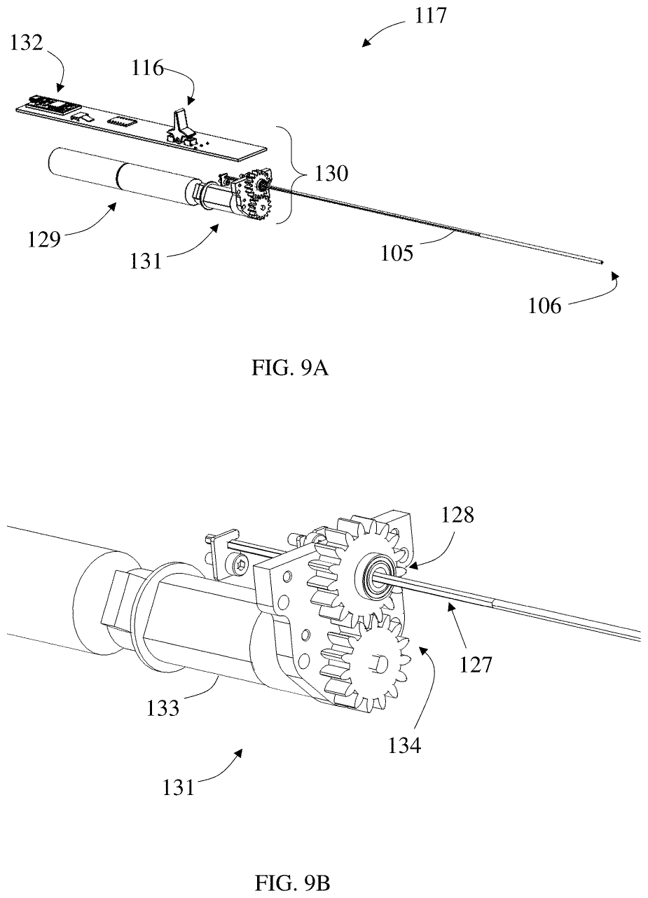

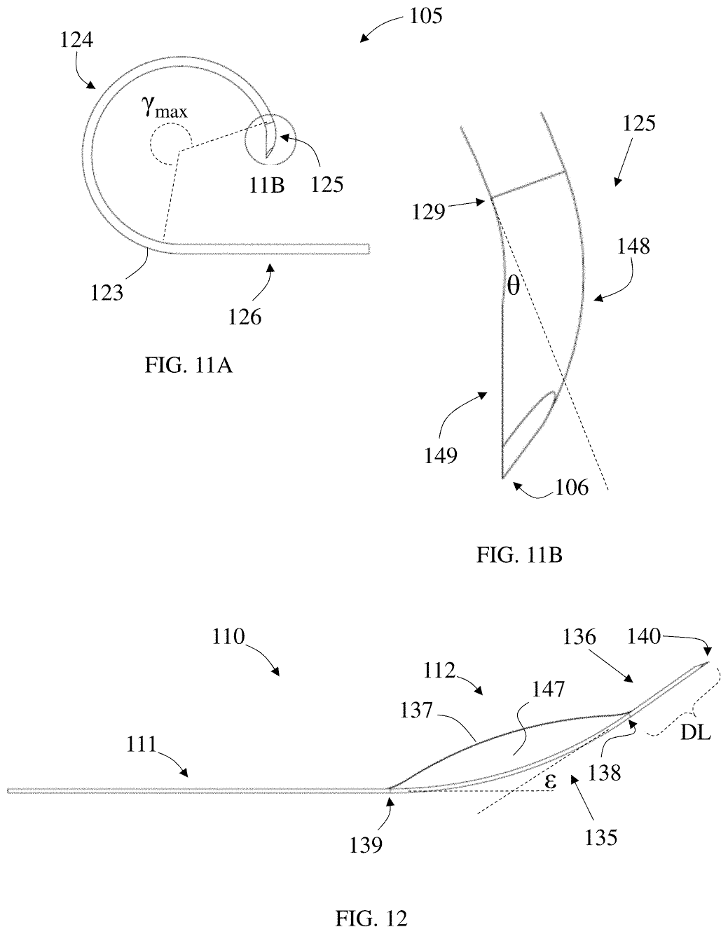

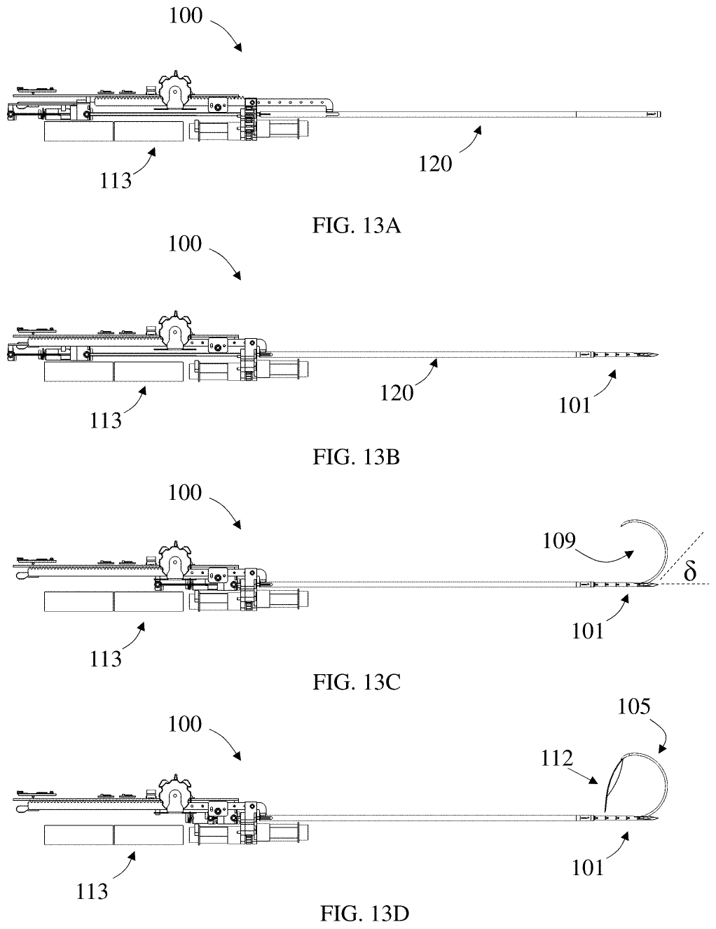

Apparatus 100 includes an inner needle 105 (shown in detail in FIGS. 9A and 11A, for example) comprising an elastic needle body 123 ending with a sharp needle tip 106 and enclosing an inner needle lumen opened to an inner needle opening (similar to inner needle lumen and opening, 58 and 59, of apparatus 50) adjacent to needle tip 106. Inner needle 105 is configured to pass straightened (as shown in FIG. 9A) through outer tube lumen 103 and to partially protrude via outer tube opening 104, such that a protruding portion 109 (shown in FIG. 13C, for example) of inner needle 105 is allowed to voluntarily flex to a curved form configured for rotationally advancing through soft tissue surrounding the tissue mass, for piercing a curved passage around the tissue mass (as described above).

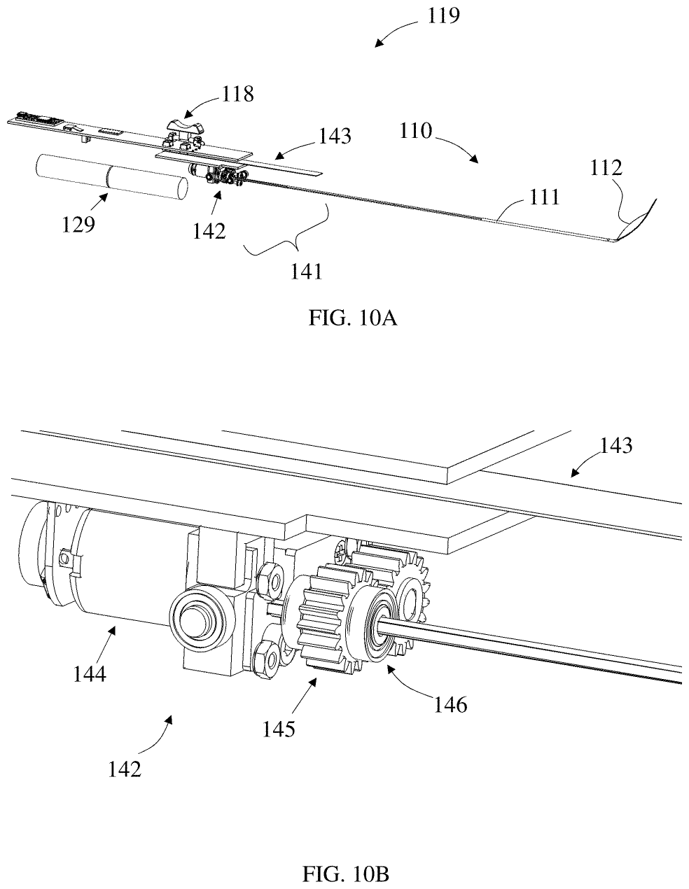

Apparatus 100 also includes a tension member passer 110 (shown in detail in FIGS. 10A and 12, for example) comprising a tension member passer body 111, sized for passing through inner needle lumen, and a tension member passer pulling portion or securing member 112 configured for securing a portion of a tension member (e.g., suture) to the tension member passer body 111.

Apparatus 100 further includes a console 113 in a form of a handheld device, and is equipped with a first control 114 formed as a knob for activating an outer tube uncovering mechanism 115 (shown in detail in FIG. 8A, for example), a second control 116 formed as a switch for activating an inner needle advancing mechanism 117 (shown in detail in FIG. 9A, for example), and a third control 118 formed as a switch for activating an inner needle advancing mechanism 119 (shown in detail in FIG. 10A, for example).

FIGS. 8A-8B illustrate respectively a full isometric view and a zoom-in partial isometric view of outer tube uncovering mechanism 115 provided in apparatus 100. Outer tube uncovering mechanism 115 is configured for fixating a chosen uncovered length 107 of outer tube 101 to cover remaining length of outer tube 101 using an outer tube cover sheath 120. Tube cover has a distal edge configured to resist penetration of outer tube 101 into soft tissue beyond the uncovered length. Outer tube 101 includes measurement readings 108 arranged to facilitate visual reading of a dimension indicative of uncovered length 107 (FIG. 8B); such readings can assist in pre-penetration preparations or be visualized from within the body, such as by a laparoscope present at a different laparoscopic entry or by an imaging equipment.

First control 114 is operatively connected to outer tube cover sheath 120 using an uncovering mechanism rack and pinion actuator 121 connected to a push rod 122 (for transmitting knob rotation motions to push rod linear motions). The knob constructed control 114 is finger-operated by forward or backward rotation to force corresponding linear motion of push rod 122 that is fixedly connected to cover sheath 120 and transmits thereto the motions applied via first control 114. Cover sheath 120 is slidable over outer tube 101 between a proximal-most position (shown in FIG. 13A), in which outer tube tip 102 is covered within cover sheath 120, and a distal-most position in which cover sheath 120 is maximally withdrawn to uncover a predetermined maximal uncovered length of outer tube 101 (as shown in FIG. 13B, for example).

FIGS. 11A-11B illustrate respectively a full side view and a zoom-in partial side view of inner needle 105 in an unstressed relaxed state (in which no external forces or internal stresses are applied in a manner sufficient to deform its size and/or shape, at least not significantly and/or visually). Inner needle 105 is configured such that when its protruding portion progresses through the soft tissue, tissue infiltration into its lumen is minimized or even prevented, and its protruded portion effectively resists straightening from its unstressed, relaxed shape. Inner needle body 123 in its unstressed relaxed state includes a flexible curved segment 124 with a radius of curvature within a range of 15 mm to 45 mm. Curved segment 124 optionally provides the maximally allowed length for protruding portion 109. When in the unstressed relaxed state, curved segment 124 forms an arc subtending an angle γₘₐₓ of at least 225°, optionally at least 270°, and is configured with elastic resistance to straightening within a range of 2 N to 20 N. When straighten, length of curved segment 124 is optionally at least 40 mm, optionally at least 100 mm, or optionally at least 250 mm.

A distal segment 125, which ends with needle tip 106, adjoins curved segment 124 with a bending 129 (optionally an inward bending, inclined towards center of curvature of curved segment 124) having a bending angle θ within a range of 5° to 25° relative to a tangent projection to curved segment 124 at bending 129. Distal segment 125 includes an outer curved side 148 and an inner straight side 149, adjoining with the sharp needle tip 106. Inner straight side 149 encloses the inner needle opening, such that the opening is positioned laterally to needle tip 106, and inwardly (at least partially towards center of curved segment 124), when inner needle 105 is pushed through soft tissue via outer tube 101. Inner needle 105, with distal segment 125 thereof, is configured such that protruding portion 109 of inner needle 105 exits outer tube opening 104 with a needle exit angle δ within a range of 10° to 80°, optionally within a range of 20° to 50°, relative to outer tube 101 (as shown in FIG. 13C). A proximal segment 126 adjoined curved segment 124, optionally straight along its length, provided with threads 127 (shown in FIG. 9B) that are configured to function as a rack member in a needle rack and pinion actuator 128.

FIGS. 9A-9B illustrate respectively a full isometric view and a zoom-in partial isometric view of inner needle advancing mechanism 117 provided in apparatus 100. Inner needle advancing mechanism 117 is powered by one or more batteries 129 and includes an inner needle protrusion controller 130 configured to operatively control advancement of inner needle 105 within outer tube 101. Inner needle protrusion controller 130 is operateable using second control 116 and includes an inner needle motion generator 131 and a needle printed circuit board 132. Second control 116 is operatively connected to inner needle motion generator 131 to selectively force axial movement of the inner needle within the outer tube and includes a needle motor 133 and a needle gear mechanism 134. By switching second control 116 in a certain direction, inner needle protrusion controller 130 comes into play and the programmed PCB 132 orders batteries 129 to power needle motor 133 in a corresponding direction. Via needle gear mechanism 134, the rotary motion is transferred to needle rack and pinion actuator 128 and transited to linear motion for shifting inner needle 105 forward or backward. Needle motion generator 131 is configured to force by default axial movement of tension member passer 110 with inner needle 105 such that both advance and withdraw together within outer tube 101. FIG. 13B shows apparatus 100 when inner needle 105 is fully retracted within outer tube 101, and FIG. 13C shows apparatus 100 when a protruding portion 109 of inner needle 105 protrudes in a curved form via outer tube opening 104.

FIG. 12 illustrates a side view of tension member passer 110 in an unstressed relaxed state (in which no external forces or internal stresses are applied in a manner sufficient to deform its size and/or shape, at least not significantly and/or visually), with its main components—tension member passer body 111 and tension member passer securing member 112—configured in a snare like form. Tension member passer body 111 is flexible and elastic and preferably solid (with no lumen extending along part or all its length).

Tension member passer 110 is configured to exit the inner needle lumen in a straight form, and optionally tangent thereto, and to keep straight when it is further advanced until a curved or bending point provided along its length reaches the inner needle opening, allowing it to incline relative to inner needle 105. Tension member passer body 111 has a curved or bent portion 135 forming a deviated tension member passer distal end portion 136, which is substantially straight and extends along a length DL which is optionally at least 10 mm, or optionally particularly between about 15 mm and about 30 mm. If, prior to tension member passer protrusion, inner needle tip 106 is distanced less than length DL from outer surface of the treated internal body region (e.g., outer surface OS), then tension member passer 110 will advance in a straight path until reaching or crossing the outer surface of the treated body region. Contrarily, if needle tip 106 is distanced substantially more than length DL, then tension member passer 110 will begin its progress in a straight form but eventually will curve and continue its advancing in a curved path. The deviated tension member passer distal end portion 136 forms with remainder of tension member passer body 111 a deviation angle £ within a range of 15° to 55°, optionally about 35°. Curved or bent portion 135 is configured with elastic resistance to straightening within a range of 0.1 N to 1 N.

Tension member passer securing member 112 includes a securing wire portion 137 extending from a first location 138 at distal end portion 136 to a second location 139 on the tension member passer body 111 proximally to curved or bent portion 135. Securing wire portion 137 is similar in length to length of a segment of tension member passer body 111 extending from first location 138 to second location 139, and form together a symmetric, elastic, normally-opened, loop 147, optionally shaped in a ‘diamond’, ‘oval’ or ‘vesica piscis’ (pointed oval) contour. Optionally, loop 147 is sized to allow passing therethrough of a grasper holding a suture, optionally having a width within a range of 2 mm to 10 mm, optionally about 5 mm. When deviated tension member passer distal end portion 136 is forced to align with rest of tension member passer body 111, securing wire portion 137 is configured to undergo increased tension, so that loop 147 compresses and can therefore hold a tension member (e.g., suture) passing therethrough. When tension member passer securing member 112 extends fully within a space of sufficient size, such as within abdominal cavity AC, loop 147 can elastically (voluntarily) expand to an open form. Optionally, securing member 112 is formed of a super elastic material, optionally Ni—Ti alloy, and/or optionally of same material as tension member passer body 111 yet in smaller width and/or different thermal conditioning. Tension member passer 110 ends with a sharp tension member passer tip 140 configured to cut through soft tissue when pressed therethrough in sufficient force.

FIGS. 10A-10B illustrate respectively a full isometric view and a zoom-in partial isometric view of tension member passer advancing mechanism 119 provided in apparatus 100. Tension member passer advancing mechanism 119 is also powered by batteries 129 and includes a tension member passer protrusion controller 141 configured to operatively control advancement of tension member passer body 111 within inner needle 105. Tension member passer protrusion controller 141 is operable using third control 118 and includes a tension member passer motion generator 142 and a tension member passer printed circuit board 143. Third control 118 is operatively connected to tension member passer motion generator 142 to selectively force axial movement of tension member passer 110 within inner needle 105. Tension member passer motion generator 142 includes a tension member passer motor 144 and a tension member passer gear mechanism 145. By switching third control 118 in a certain direction, tension member passer protrusion controller 141 comes into play and the programmed tension member passer PCB 143 orders batteries 129 to power tension member passer motor 144 in a corresponding direction. Via tension member passer gear mechanism 145, the rotary motion is transferred to a tension member passer rack and pinion actuator 146 and translates to linear motion for shifting tension member passer 110 forward or backward relative to inner needle 105. Tension member passer PCB 143 can be configured such that tension member passer protrusion controller 141 can be activated to advance tension member passer body 111 only after needle protrusion controller 130 finishes advancing of inner needle 105 up to a user defined length of protruding portion 109. FIG. 13C shows apparatus 100 when tension member passer 110 is fully retracted within inner needle 105, and FIG. 13D shows apparatus 100 when tension member passer securing member 112 protrudes from inner needle 105.

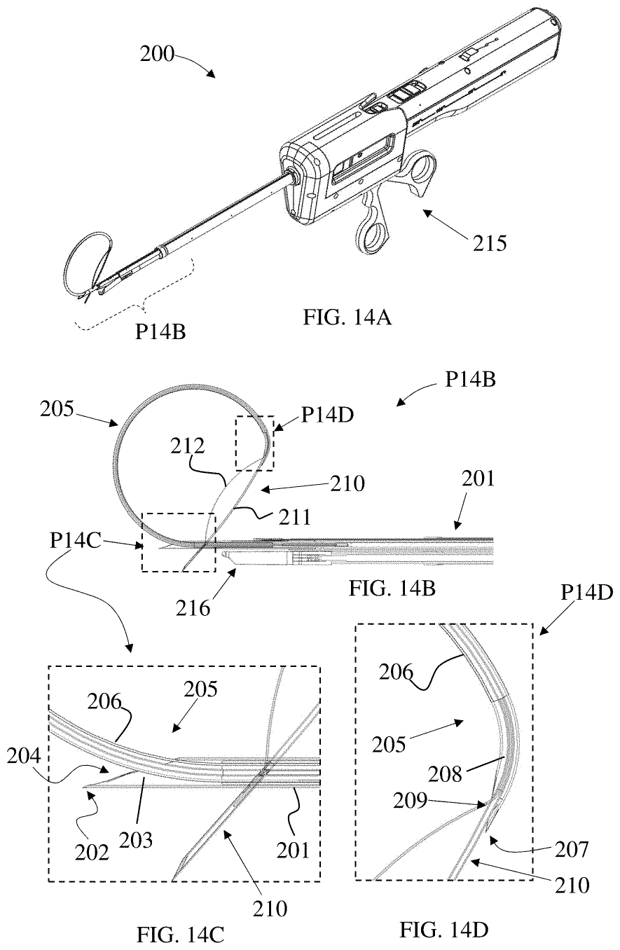

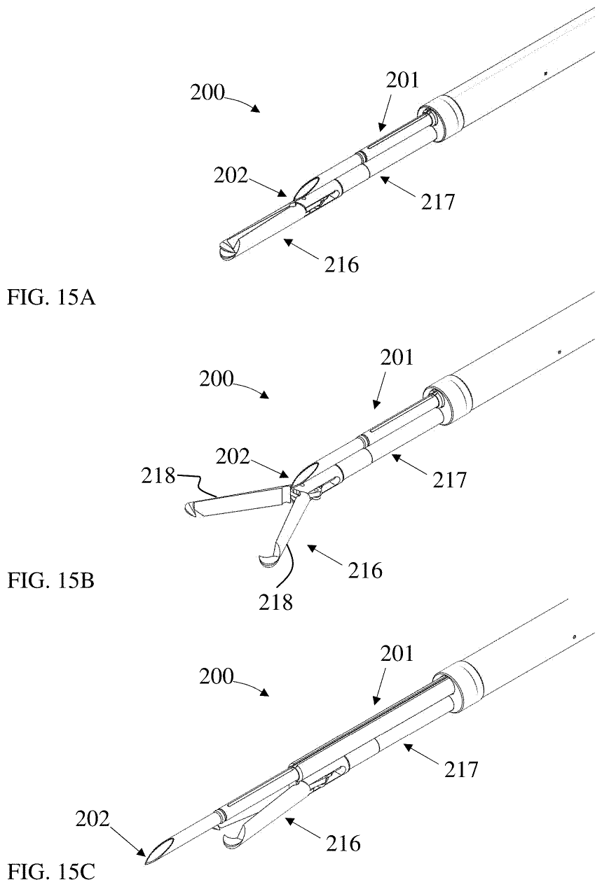

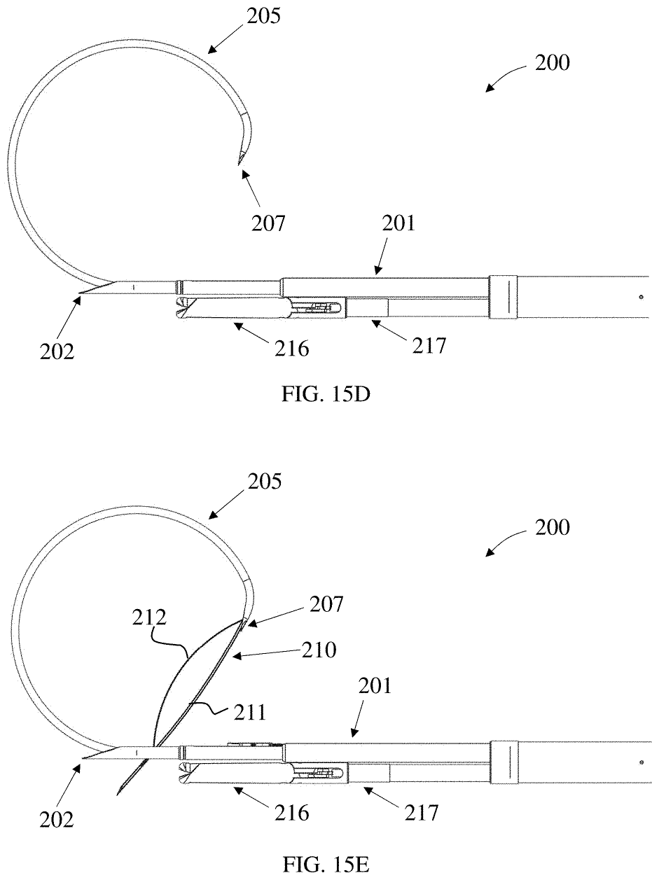

FIGS. 14A-14D illustrate views of an exemplary apparatus 200 for passing a tension member around a chosen volumetric region of an organ and/or a tissue mass in the organ. FIG. 14B shows a side cross sectional view of a distal (front) portion P14B of apparatus 200 shown in FIG. 14A; FIG. 14C shows a side cross sectional view of portion P14C of portion P14B shown in FIG. 14B; and FIG. 14D shows a side cross sectional view of portion P14D of portion P14B shown in FIG. 14B. FIGS. 15A-15E illustrate views of a distal portion of apparatus 200 which represent several exemplary scenarios of its operation.

Apparatus 200 is optionally an exemplary implementation or variation of apparatus 50 and/or apparatus 100 described above, and it may include some or all embodiments and features of apparatus 50 and/or apparatus 100. Similarly, apparatus 200 is configured for forming a passage through an organ surrounding the volumetric region and/or tissue mass, from an entry point at a surface of the organ to an opposingly located exit point at the surface of the organ, relative to the tissue mass, and for passing the tension member around the volumetric region and/or tissue mass by pulling the tension member from the exit point to the entry point through the passage. In addition to features described with respect to apparatus 100, for example, apparatus 200 also employs a forceps head 216 and mechanism for using it to hold a portion of the treated organ in a chosen distance, relative to a fixed portion of apparatus 200, while forming a surgical passage in the organ and/or passing the tension member through the surgical passage.

Apparatus 200 is optionally a handheld device, although it can—as a whole or in part—be integrated in a robotic system; and it includes a plurality of members, devices and/or mechanisms housed in, connected to, and/or operational via, a console 215. Apparatus 200 includes a substantially straight rigid outer tube 201 comprising a sharp outer tube tip 202 and an outer tube lumen 203 with an outer tube opening 204 in proximity (e.g., adjacent) to the outer tube tip 202. Apparatus 200 also includes an inner needle 205 which comprises an elastic needle body 206, curved at least in part thereof and configured with elastic resistance to straightening within a range of 2 N to 20 N. Needle body 206 ends with a sharp needle tip 207 and encloses an inner needle lumen 208 with an inner needle opening 209 in proximity (e.g., adjacent) to the needle tip 207. Inner needle body 206 is configured to pass straightened through outer tube lumen 203 and to partially protrude via the outer tube opening, such that a protruding portion thereof is allowed to voluntarily flex to a curved form having diameter equal to or greater than diameter of the target tissue mass.

Apparatus 200 also includes a tension member passer 210 which is similar or identical to tension member passer 110 shown in FIG. 12. Tension member passer 210 includes a tension member passer body 211, which is flexible and elastic and sized for passing through inner needle lumen 208. Tension member passer 210 also includes a tension member pulling portion configured for engaging with a portion of the tension member and for continuously applying a pulling force to the engaged portion of the tension member when the tension member is withdrawn with the tension member passer. In some embodiments, and as shown, tension member pulling portion includes a tension member passer securing member 212 which may include or be configured as any member or mechanism capable of automatically or selectively (e.g., manually) capturing a portion of a tension member (e.g., in a form of a wire or a suture) with sufficient force to draw the tension member through the tissue and/or the premade passage made through the tissue, within the organ or body region BR and/or the tumor or tissue mass TM.

In the illustrated embodiment, securing member 212 comprises a resiliently deformable member that forms a loop with the tension member passer body 211. Securing member 212 is configured for securing a portion of the tension member to apparatus 200 when tension member passer body 211 is withdrawn into inner needle body 206 in a way that the loop surrounding the tension member gradually withdrawn into inner needle lumen 208 while its remainder portion outside inner needle lumen 208 increases embracing and tightening around the held portion of the tension member. As such, the securing member 212 secures the portion of the tension member to tension member passer body 211 and/or to inner needle body 206. Some or all other structural and/or functional features of tension member passer 110 described above are also applicable to tension member passer 210.

Forceps head 216 is fixedly connected to console 215 with a forceps shaft 217. Forceps shaft 217 extends distally from the console, adjacent and aligned parallel to the outer tube 201. Forceps head 216 is fixedly positioned distally to the console 215 and is activatable via the console and configured for selective grasping of the treated organ adjacent to the entry point and/or the exit point, for holding the grasped organ at a fixed distance relative to console 215. Forceps head 216 is configured in a form of tenaculum having two hinged tenaculum arms 218, each tenaculum arm includes a slender sharp-pointed hook configured to penetrate through the organ surface into the organ when forceps head 216 is operated to grasp the organ surface. After the organ is grasped with forceps head 216, outer tube 201 is allowed to slide distally relative forceps head 216 such that outer tube tip 202 can extend distally beyond forceps head 216 and penetrate into the organ (and form the entry point thereinto). In some embodiments, apparatus 200 includes a safety mechanism that prevents forward extension of outer tube 201 before forces head 216 is operated and/or grasps the organ, and this safety mechanism operates either manually or automatically. Optionally, apparatus 200 is configured to prevent extension of outer tube tip 202 over a predetermined maximal penetration depth beyond forceps head 216.

Apparatus 200 can be applied for passing one or more tension members around a tissue mass such as a tumor (e.g., fibroid), as described above. A plurality of tension members (e.g., in a form of wire or suture), distributed along spaced paths around the tissue mass, for example, can be applied with controlled and/or continuous tension therethrough, thereby collectively apply spatial inward compression of the tissue mass with the taut tension members. This method was found to cause ischemia and/or necrosis to target tissue masses under prolonged spatial compressions. In order to pass a tension member, a surgical passage is first created in a bodily organ (e.g., uterus), around a target tissue mass (e.g., tumor or portion thereof) and then a tension member is passed in reversed travel through the passage. Tension applied to the one or more tension members after implantation thereof, can be performed using apparatus 200, using another device, or performed manually.

A method for passing tension member around tissue mass may include some or all steps of the method shown in FIGS. 6A-6H, for example, although it may include additional and/or different steps, or variations thereof. One or more of the following exemplary steps can be performed (not necessarily in the listed order) in order to form a surgical passage followed by tension member passing around a target tissue mass in an organ:
Reaching the surface of the organ inside subject's body: forming a minimally invasive surgical route from outside subject's body to the (internal) organ, and delivering the distal (front) portion of apparatus 200 through the surgical rout until the distal portion of apparatus 200 reaches a chosen surface portion of the organ (similarly to as shown in FIGS. 6A-6B), for example), and the operator can verify that apparatus 200 can be applied to pass a tension member within the organ as needed. FIG. 15A shows distal portion of apparatus 200 in a pre-deployment configuration, such as during delivery via the surgical rout to the organ surface, with forceps head 216 in a closed formation and outer tube 201 fully withdrawn such that tip 202 thereof is positioned proximally to forceps head 216.
Fixating the organ penetration area: using forceps head 216, grasping the organ adjacent to the area where the entry point and/or the exit point will be formed, for holding the grasped organ before the penetrating with the outer tube. FIG. 15B shows distal portion of apparatus 200 with forceps head 216 in a fully opened configuration before grasping, while FIG. 15C shows a closed or grasping state of forces head 216 (the grasped organ is not shown in this figure).
Penetrating the organ: penetrating into the organ using rigid outer tube 201 of apparatus 200 such that outer tube tip 202 reaches a chosen, optionally predetermined, penetration depth (similarly to as shown in FIG. 6B, for example). As shown in FIG. 15C, outer tube 201 is advanced distally up to a full extension, such that tip 202 thereof is positioned at a chosen length distally to forceps head 216, optionally within a range of about 0.5 cm to about 3 cm, for example. Since that the organ is held fixed at a constant distance relative to console 215, adjacent to the entry point to the organ formed by outer tube tip 202 due to its shift forward, the penetration characteristics (e.g., resistance to penetration to the organ, penetration depth and orientation, etc) are controlled and able to be predetermined to high degree.
Extending the passage in the organ to surround the target tissue mass: passing inner needle 205 in outer tube lumen 203 followed by piercing a curved passage with needle tip 207 around the tissue mass with a protrusion length of a protruding portion of the inner needle body, by pushing the inner needle 205 via the outer tube opening 204 and allowing the protruding portion to voluntarily flex to a curved form, optionally having diameter equal to or greater than diameter of the tissue mass, until distal portion of inner needle 205 is positioned and oriented in a chosen location in the organ (similarly to as shown in FIG. 6C, for example). As shown in FIG. 15D, inner needle 205 is pushed up to a chosen extent (length and/or radius, for example) and allowed to flex to a curved form determined by its relaxed size and/or shape and possibly also resistance to advancement and/or flexion applied to portions thereof by the surrounding tissue.
Further extending the passage until exiting the organ: advancing tension member passer 210 in the inner needle lumen and via the inner needle opening, until tension member passer securing member 212 exits the organ at an exit point opposing the entry point relative to the tissue mass (similarly to as shown in FIG. 6D, for example). As shown in FIG. 15E, once inner needle tip 207 is positioned in a chosen (e.g., predetermined or determined in progress, for example) location and/or orientation (within the organ, relative to the target tissue mass, distance to organ surface and/or entry point thereto, for example), tension member passer 210 is advanced via inner needle 205 to a chosen extent until securing member 212 or portion thereof exits the organ at the exit point formed by the distal tip of tension member passer 210.
Passing a tension member around the target tissue mass along the surgical passage: securing a portion of a tension member to tension member passer body 210 and/or inner needle body 206, and drawing the tension member into the inner needle lumen 208 by pulling tension member passer 210 with the tension member secured using the securing member 212 (similarly to as shown in FIGS. 6E-6F, for example). The drawing may include extending the tension member around the tissue mass such that one end of the tension member projects from the entry point and another end of the tension member projects from the exit point (similarly to as shown in FIG. 6G, for example).
Optionally repeating some or all steps for surrounding the target tissue mass, optionally with spatially spaced passages and corresponding tension members extended therethrough, using apparatus 200 or a similar apparatus, optionally with same or different sized or shaped outer tube, inner needle and/or tension member passer.

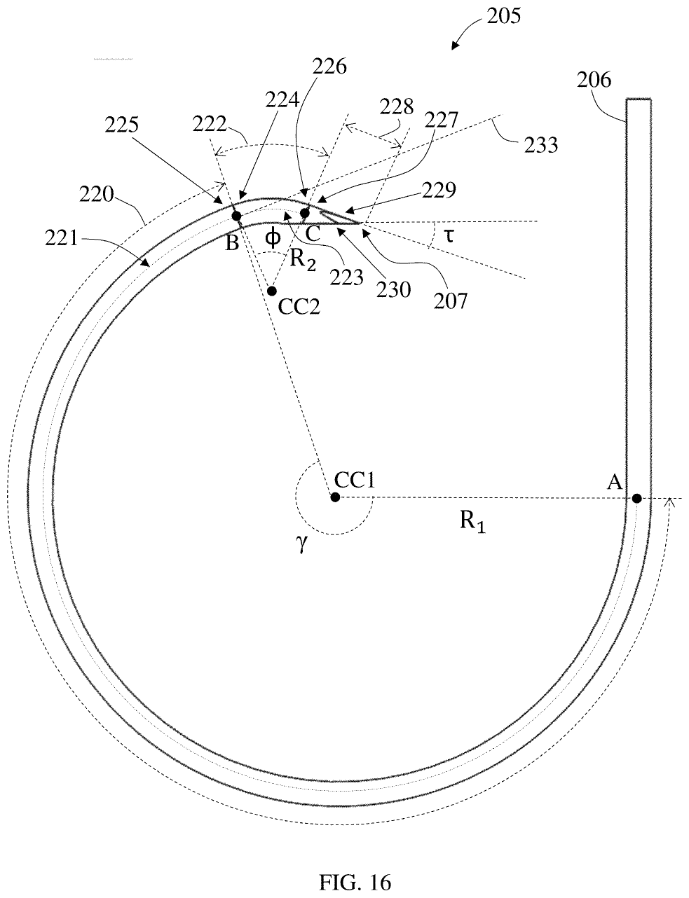

FIG. 16 illustrates a side view of an exemplary surgical needle applicable and configured as inner needle 205 of apparatus 200. Needle 205 is optionally an exemplary implementation or variation of needle 105 described above and shown in FIGS. 11A-11B, and it may include some or all embodiments and features of needle 105. As previously described, needle 205 includes elastic needle body 206 ending with sharp needle tip 207. Needle body 206 is configured with elastic resistance to straightening within a range of 2 N to 20 N, and includes a first segment 220 (extending between points A and B as shown) having a first centerline 221, and a second segment 222 (extending between points B and C as shown) having a second centerline 223. Second segment 222 adjoins with a proximal end 224 thereof to a distal end 225 of first segment 220 and with a distal end 226 thereof to a proximal end 227 of a tip segment 228.

When the needle body 206 is in an unstressed relaxed form (as shown in FIG. 16), first centerline 221 has a first radius of curvature R1, at least adjacent to first segment distal end 225, and second centerline 223 has a second radius of curvature R2, at least adjacent to second segment proximal end 224, smaller than first radius of curvature R1. Optionally and as shown, first radius of curvature R1 is substantially constant along most or all part of first segment 220 and equals its distance from a singular first center of curvature CC1. Similarly, second radius of curvature R2 may be substantially constant along most or all part of second segment 222 and equals its distance from a singular second center of curvature CC2. Optionally, first radius of curvature R1 is within a range of 15 mm to 45 mm when needle body 206 is in the unstressed relaxed state. The ratio R2:R1 between second radius of curvature R2 and first radius of curvature R1 is smaller than ½, optionally within a range of 1/10 to ⅓, optionally between about 1/7 and about ⅕. First segment 220 forms a first outer arc subtending a first subtended angle γ within a range of 200° to 300°, optionally between 240° and 260°. Second segment forms a second outer arc subtending a second subtended angle φ, smaller than first subtended angle γ, within a range of 10° to 80°, optionally between 30° and 60°.

Tip segment 228 has a tridimensional beveled shape formed by adjoined curved outer surface 229 and flat inner surface 230 intersecting at the needle tip 207. Inner surface 230 is tilted radially inwardly relative to a forward tangent 233 of first segment distal end 225. A (straight) outline of outer surface 229 extends from second segment distal end 226 above (radially outwardly to) point C of second centerline 223. A (straight) outline of inner surface 230 extends from a mid-portion of second segment 222 below second centerline 223. The outline of outer surface 229 is shorter than the outline of inner surface 230, optionally about half in length thereof. An intersection angle r, defined by intersection of outer surface 229 and inner surface 230 at needle tip 207, is within a range of 10° to 30°, optionally about 20°.

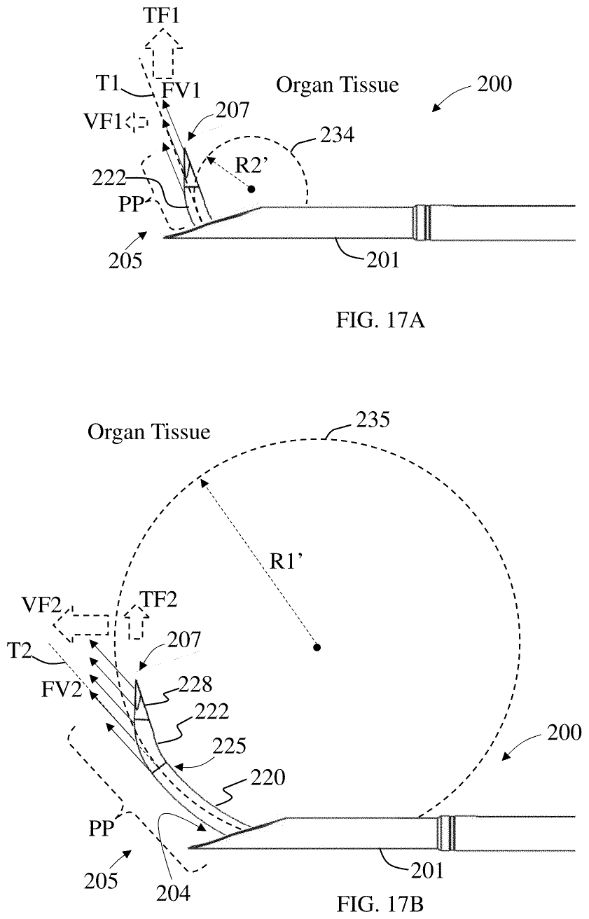

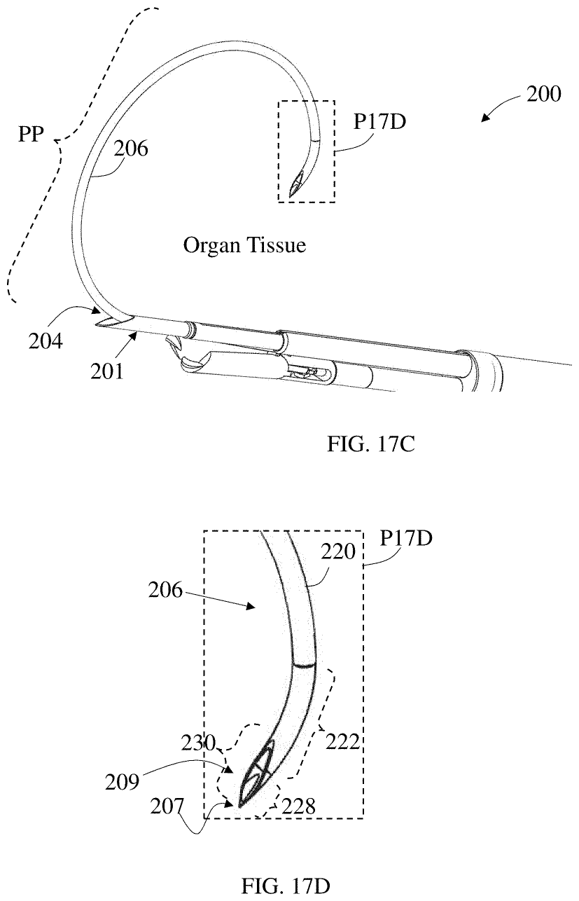

FIGS. 17A-17D illustrate a distal portion of apparatus 200 representing several scenarios of advancing needle 205. As shown, when inner needle body 206 begins to protrude via outer tube opening 204, a protruding portion PP of the inner needle body 206 is allowed to voluntarily flex to a curved form. As long as protruding portion PP includes only second segment 222 or part thereof, thereby it excludes first segment distal end 225, inner needle tip 207 is configured to follow a first curved path 234 having a radius of curvature R2′ which is similar (e.g., substantially equal, or slightly smaller or greater than) to second radius of curvature R2 (FIG. 17A). When the protruding portion PP exceeds the length of second segment 222 to include also at least first segment distal end 225, the protruding portion PP shifts into following a second curved path 235 having a radius of curvature R1′ (FIG. 17B). In some embodiments, radius of curvature R1′ is similar (e.g., substantially equal, or slightly smaller or greater than) to or smaller than first radius of curvature R1.

Alternatively, radius of curvature R1′ may be greater than radius of curvature R1, however the integration or merging of first and second curved paths 234 and 235, such that second curved path 235 begins from first curved path 234, forms an integrated or complete path of protruding portion PP at its full extent, optionally having an average or median radius of curvature being similar to or smaller than first radius of curvature R1. In some such embodiments, the introduction of a first curved segment having smaller radius of curvature affects a preliminary curved path intended to diminish and/or compensate for increase of average or median radius of curvature, relative to first radius of curvature R1, as a result of (radial) resistance to penetration from the surrounding organ tissue. Optionally, additionally or alternatively, by causing a preliminary penetration having a substantially smaller radius of curvature, the lateral (side) penetration from outer tube 201 is easier with less resistance by the surrounding organ tissue and/or harm thereto.

As shown in FIG. 17A, during initial advancement of protruding portion PP with radius of curvature R2′, a first force vector FV1 transferred through first segment 220 to second segment 222 and tip segment 228, and applied from tip segment 228 to surrounding organ tissue, approximates a tangent T1 to first curved path 234, therefore tangential force component TF1 of first force vector FV1 is substantially greater than normal force component NF1 of first force vector FV1 resulting in more efficient transfer of force-to-penetration with relatively less lateral resistance affected by surrounding tissue.

On the other hand, and as demonstrated in FIG. 17B, during further advancement of protruding portion PP with radius of curvature R1′, a second force vector FV2 transferred through first segment 220 to second segment 222 and tip segment 228, and applied from both second segment 220 and tip segment 228 to surrounding organ tissue, approximates a tangent T2 to second curved path 235. Therefore, a normal force component NF2 of second force vector FV2 is greater than normal force component NF1 of first force vector FV1. This may result in increased lateral resistance affected by surrounding tissue, optionally forcing protruding portion PP to follow second curved path 235 with radius of curvature R1′ smaller than first radius of curvature R1. In some embodiments, tangential force component TF2 is substantially smaller than normal force component NF2 of second force vector FV2

In some embodiments, needle body 206 is shaped and configured such that the radial force (resisting advancement) applied by surrounding tissue increases during the further advancement of protruding portion PP in the organ such that radius of curvature R1′ of second curved pass 235 reduces per further advancement relative to first radius of curvature R1 of needle body's first centerline 221 when in the unstressed relaxed form.

FIG. 17C shows an isometric view of a distal portion of apparatus 200 with needle 205 emerging closer to a full extent, in an angle of view that reveals inner needle opening 204. FIG. 17D is a zoom-in view of portion P17D of FIG. 17C. As shown, inner needle opening 209 is provided in proximity to needle tip 207, surrounded with flat inner surface 230 extending from about mid length of second segment 222 and across tip segment 228. Due to shape of needle body 206, when the protruding potion PP advances through the organ tissue, penetration through additional incoming segment of organ tissue is achieved by forcing curved outer surface 229 against it, in direction of first force vector FV1 or second force vector FV2, such that inner surface 230 and needle opening 209 are concealed (also as demonstrated in FIG. 17B, for example, showing the side of needle opening 209 in opposite direction to direction of the force vectors FV1 and FV2). This way, coring (i.e., undesired entrapment or entering of tissue into inner needle lumen 203 via needle opening 209) of a currently penetrated segment of the organ tissue, via inner needle opening 209, is prevented.

### EXAMPLES

Following are various illustrative examples, each of which is a separate embodiment. This disclosure further includes all permutations of the “independent” examples below with their “dependent” examples. Moreover, additional embodiments capable of derivation from the independent and dependent examples that follow are also expressly incorporated into the present written description.

### Example 1

An apparatus for passing a tension member around a volumetric portion of an organ can comprise: a rigid outer tube comprising a sharp outer tube tip and an outer tube lumen with an outer tube opening in proximity to the outer tube tip; an inner needle comprising an elastic needle body curved at least in part thereof, the inner needle ending with a sharp needle tip and enclosing an inner needle lumen with an inner needle opening being in proximity to the needle tip, the inner needle body being configured to pass straightened through the outer tube lumen and to partially protrude via the outer tube opening, such that a protruding portion of the inner needle body is allowed to voluntarily flex to a curved form having diameter equal to or greater than diameter of the volumetric portion; and a tension member passer comprising a tension member passer body, sized for passing through the inner needle lumen, and a tension member pulling portion configured for engaging with a portion of the tension and for continuously applying a pulling force to the engaged portion of the tension member when the tension member is withdrawn with the tension member passer; wherein the apparatus is configured for forming a passage through the organ, the passage extending along a plane crossing the volumetric portion from an entry point at a surface of the organ, located in front of a first side of the volumetric portion, to an exit point at the surface of the organ, located in front of a second side of the volumetric portion opposite to the first side, and the apparatus is further configured for passing the tension member around the volumetric portion by pulling the tension member from the exit point to the entry point through the passage.

In various embodiments, the volumetric portion of the organ includes a tissue mass comprising at least a portion of a tumor.

In various embodiments, the outer tube is movable relative to a covering portion of the apparatus until the outer tube tip extends a chosen uncovered length from a distal edge of the covering portion, the distal edge is configured to resist penetration into soft tissue to inhibit insertion of the outer tube to a depth greater than the uncovered length. In various embodiments, the apparatus comprising measurement readings arranged to facilitate visual reading of a dimension indicative of the uncovered length.

In various embodiments, the outer tube opening is located proximally to the outer tube tip at a side of the outer tube.

In various embodiments, the outer tube opening is located adjacent to the outer tube tip.

In various embodiments, the needle body includes a first segment having a first centerline, and a second segment having a second centerline, the second segment adjoins with a proximal end thereof to a distal end of the first segment and with a distal end thereof to a proximal end of a tip segment, wherein, when the needle body is in a relaxed form, the first centerline has a first radius of curvature, at least along a portion thereof being adjacent to the first segment distal end, and the second centerline has a second radius of curvature, at least along a portion thereof being adjacent to the second segment proximal end, the second radius of curvature is smaller than the first radius of curvature.

In various embodiments, a ratio between the second radius of curvature and the first radius of curvature is within a range of 1/10 to ⅓, optionally between about 1/7 and about ⅕. In various embodiments, when the needle body is in the relaxed form, the first segment subtends a first subtended angle and/or the second segment subtends a second subtended angle, wherein the second subtended angle is smaller than the first subtended angle. In various embodiments, the first subtended angle is within a range of 200° to 300°, optionally between 240° and 260°, and/or the second subtended angle is within a range of 10° to 80°, optionally between 30° and 60°.

In various embodiments, the tip segment has a tridimensional beveled shape formed by adjoined curved outer surface and flat inner surface intersecting at the needle tip, wherein outline of the outer surface extends from a point of the second segment distal end located radially outwardly to the second centerline and outline of the inner surface extends from a point between the second segment proximal end and second segment distal end located radially inwardly to the second centerline, relative to the second radius of curvature. In various embodiments, the inner surface surrounds the inner needle opening. In various embodiments, an intersection angle defined by intersection of the outer surface and the inner surface at the needle tip is within a range of 10° to 30°, optionally about 20°. In various embodiments, the outline of the outer surface is straight. In various embodiments, the outline of the outer surface is shorter than the outline of the inner surface, optionally about half in length thereof. In various embodiments, the inner surface is tilted radially inwardly relative to a forward tangent of the first segment distal end.

In various embodiments, the first radius of curvature is within a range of 15 mm to 45 mm when the needle body is in the unstressed relaxed state.

In various embodiments, the elastic needle body is configured with elastic resistance to straightening within a range of 2 N to 20 N.

In various embodiments, the apparatus is configured such that the protruding portion exits the outer tube opening with a needle exit angle δ within a range of 10° to 80°, optionally within a range of 20° to 50°, relative to the outer tube.

In various embodiments, the tension member passer body is flexible and elastic.

In various embodiments, the tension member passer body is solid.

In various embodiments, the tension member pulling portion includes a securing member forming a loop with the tension member passer body.

In various embodiments, the tension member passer body has a curved or bent portion forming a deviated distal end portion inclined relative to remainder of the tension member passer body. In various embodiments, the deviated tension member passer distal end portion forms with rest of the tension member passer body a deviation angle within a range of 15° to 55°, optionally about 35°.

In various embodiments, the tension member pulling portion includes a securing wire portion extending from a first location on the tension member passer body, distally to the curved or bent portion, to a second location on the tension member passer body, proximally to the curved or bent portion. In various embodiments, the securing wire portion is similar in length to length of a segment of the tension member passer body extending from the first location to the second location. In various embodiments, the securing wire portion is configured to undergo increased tension when the deviated tension member passer distal end portion is forced to align with rest of the tension member passer body. In various embodiments, the deviated tension member passer distal end originates at the first location and extends in a straight form at least 10 mm in length. In various embodiments, the curved or bent portion of the tension member passer body is configured with elastic resistance to straightening within a range of 0.1 N to 1 N.

In various embodiments, the apparatus further comprising a console, optionally formed as a handheld device.

In various embodiments, the apparatus further comprising an inner needle protrusion controller configured to operatively control advancement of the inner needle within the outer tube. In various embodiments, the inner needle protrusion controller includes a second control operatively connected to an inner needle motion generator configured to selectively force axial movement of the inner needle within the outer tube. In various embodiments, the needle motion generator includes at least one of a needle motor, a needle gear mechanism and a needle printed circuit board. In various embodiments, the needle motion generator is configured to force by default axial movement of the tension member passer with the inner needle such that both advance and/or withdraw together within the outer tube.

In various embodiments, the apparatus further comprising a tension member passer protrusion controller configured to operatively control advancement of the tension member passer body within the inner needle. In various embodiments, the tension member passer protrusion controller is operable using a third control operatively connected to a tension member passer motion generator configured to selectively force axial movement of the tension member passer within the inner needle. In various embodiments, the tension member passer motion generator includes at least one of a tension member passer motor, and a tension member passer gear mechanism. In various embodiments, the apparatus is configured such that the tension member passer protrusion controller can be activated to advance the tension member passer body only after the needle protrusion controller finishes advancing of the inner needle up to a user defined length of the protruding portion.

In various embodiments, the apparatus further comprising a forceps head fixedly positioned distally to the console and activatable via the console, configured for selective grasping of the organ adjacent to the entry point and/or the exit point, for holding the grasped organ at a fixed distance relative to the console. In various embodiments, the forceps head is configured in a form of tenaculum having two hinged tenaculum arms, each tenaculum arm includes a slender sharp-pointed hook configured to penetrate through the organ surface into the organ when the forceps head is operated to grasp the organ surface. In various embodiments, the outer tube is slidable distally relative to the forceps head such that the outer tube tip is extendable distally beyond the forceps head. In various embodiments, the apparatus is configured to prevent extension of the outer tube tip distally beyond the forceps head over a predetermined maximal penetration depth. In various embodiments, the forces head is connected to the console with a forceps shaft extending from the console adjacent and aligned parallel to the outer tube.

### Example 2

A method for passing a tension member around a volumetric portion of an organ can comprise: using a rigid outer tube, comprising a sharp outer tube tip and an outer tube lumen with an outer tube opening in proximity to the outer tube tip, penetrating into the organ such that the outer tube tip reaches a penetration depth; passing an inner needle in the outer tube lumen, the inner needle includes an elastic needle body curved at least in part thereof, ending with a sharp needle tip and enclosing an inner needle lumen with an inner needle opening in proximity to the needle tip; piercing a curved passage with the needle tip around the volumetric portion with a protrusion length of a protruding portion of the inner needle body, by pushing the inner needle via the outer tube opening and allowing the protruding portion to voluntarily flex to a curved form having diameter equal to or greater than diameter of the volumetric portion; advancing a tension member passer comprising a tension member passer body and a tension member pulling portion, in the inner needle lumen and via the inner needle opening, until the tension member pulling portion exits the organ at an exit point opposing the entry point relative to the volumetric portion; and drawing the tension member into and through the curved passage by pulling the tension member passer with the secured tension member.

In various embodiments, the drawing includes extending the tension member around the volumetric portion such that one end of the tension member projects from the entry point and another end of the tension member projects from the exit point.

In various embodiments, the organ is an internal organ located within a body of a live subject, and the method further comprising forming a surgical route from outside the body of the subject and delivering the outer tube through the surgical route until the outer tube tip reaches the organ. In various embodiments, the organ is a uterus.

In various embodiments, the volumetric portion of the organ includes a tissue mass comprising at least a portion of a tumor.

In various embodiments, the method comprising ending the piercing with positioning the needle tip at a chosen distance from the surface of the organ, so as to form a needle tip angle between the needle tip and the surface of the internal body region. In various embodiments, the distance is smaller than 3 cm.

In various embodiments, the needle tip angle is within a range of 10° to 60°.

In various embodiments, the defining includes defining a penetration angle between the outer tube and a perpendicular line to the surface of the internal body region at the entry point, wherein the protrusion length subtends a subtended angle is at least 270° minus the penetration angle.

In various embodiments, the penetrating, the passing, the piercing, the advancing and/or the securing is repeated, each repetition is performed using a different implanted tension member, a different entry point and a different exit point.

In various embodiments, the method can comprise: using a forceps head, grasping the organ adjacent to the entry point and/or the exit point for holding the grasped organ before the penetrating with the outer tube. In various embodiments, the outer tube is slidably connected to a console and the forceps head is fixedly positioned distally to the console, wherein the outer tube is slidable distally relative to the forceps head such that the outer tube tip is extendable distally beyond the forceps head up to a predetermined maximal penetration depth.

### Example 3

An apparatus for passing a tension member around a tissue mass that is positioned below a surface of an organ, the surgical apparatus can comprise: a tube comprising a tube lumen, a tube opening at a distal end of the tube, and a tube tip configured to penetrate through the surface of the organ to form an entry point and a passage into the organ; a needle comprising a needle lumen, a needle tip configured to penetrate tissue of the organ, and a needle body of which at least a distal portion is configured to transition between a straightened orientation and a curved orientation, the distal portion of the needle being configured to: be received within the tube lumen in the straightened orientation, pass through the tube opening and out of the tube lumen as the needle is advanced distally relative to the tube, and automatically transition to the curved orientation upon passing through the outer tube opening so as to extend the passage through the organ along a curved path that extends around at least a portion of the tissue mass; and a tension member passer configured to be advanced distally through the needle lumen and couple with the tension member; wherein at least one of the needle and the tension member passer is configured to be advanced distally relative to the tube to extend the pathway back through the surface of the organ at an exit point from the organ, and wherein the tension member passer is configured to couple to the tension member at a position external to the organ to then pass the tension member through the exit opening, then through the passage, and then through the entry opening.

In various embodiments, the distal portion of the needle is in a stressed state when in the straightened orientation and is in a relaxed state when in the curved orientation.

In various embodiments, the distal portion of the needle is deformed from a natural state into the straightened orientation when received within the tube lumen, and wherein the distal portion of the needle resiliently returns to the natural state when transitioned to the curved orientation.

In various embodiments, when the distal portion of the needle is positioned within the outer tube, the outer tube maintains the distal portion of the needle in the straightened orientation against a resilient bias that tends to return the distal portion of the needle to the curved orientation.

In various embodiments, the distal portion of the needle is further configured to be retracted through the tube opening and back into the tube lumen when the needle is retracted proximally relative to the tube.

In various embodiments, the distal portion of the needle is configured to automatically transition from the curved orientation to the straightened orientation as the needle is retracted proximally relative to the tube.

In various embodiments, the apparatus further comprising an actuator configured to distally advance the needle relative to the tube.

In various embodiments, the actuator is further configured to proximally retract the needle relative to the tube.

In various embodiments, the actuator is configured to remain at an exterior of a patient throughout usage of the apparatus.

In various embodiments, the apparatus further comprising an additional actuator configured to distally advance the tension member passer relative to the needle.

In various embodiments, the actuator and the additional actuator are configured to remain at an exterior of a patient throughout usage of the apparatus.

In various embodiments, the actuator is further configured to proximally retract the needle relative to the tube, and wherein the additional actuator is further configured to retract the tension member passer relative to the needle.

In various embodiments, the apparatus further comprising a covering coupled to the tube so as to be stationary relative to the tube, wherein the covering is configured to be selectively advanced or retracted relative to the tube to define an uncovered length of the tube.

In various embodiments, the covering comprises a blunt distal end that is resistant to passage through the surface of the organ such that the covering resists advancement of the tube into the organ beyond the uncovered length of the tube.

In various embodiments, the apparatus further comprising an actuator configured to adjust a position of the covering relative to the tube.

In various embodiments, the tube comprises a plurality of depth markings that can assist in adjusting the uncovered length of the tube to a desired length.

In various embodiments, the tube comprises a plurality of depth markings.

In various embodiments, the tube opening is at a distalmost tip of the tube.

In various embodiments, the tube opening is at a side of the tube.

In various embodiments, the tube comprises a sidewall, and wherein the tube opening extends through the sidewall.

In various embodiments, the tube further comprises a ramp configured to urge the needle tip laterally through the tube opening at the side of the tube.

In various embodiments, the ramp is at a distal end of the tube lumen.

In various embodiments, the apparatus further comprising the tension member.

In various embodiments, the tension member comprises a suture.

In various embodiments, the tension member comprises a wire.

In various embodiments, the tension member passer comprises a tension member passer body configured to follow a curvature of the needle when the needle is in the curved orientation.

In various embodiments, the distal portion of the tension member passer is configured to define a curve of a larger radius of curvature or to extend substantially rectilinearly when the distal portion of the tension member passer is advanced distally past the needle tip.

In various embodiments, the tension member passer body is resiliently deformable.

In various embodiments, the tension member passer comprises an attachment mechanism configured to couple the tension member to the tension member passer.

In various embodiments, the attachment mechanism defines a loop that is expanded when the attachment mechanism is advanced distally beyond the needle tip and is contracted when the attachment mechanism is retracted proximally into the needle.

In various embodiments, the tension member passer is configured to be advanced distally through the needle lumen to expose the attachment mechanism at an exterior of the needle, and the tension member passer is configured to be attached to the tension member when exposed at the exterior of the needle.

In various embodiments, the tension member passer comprises a tension member passer body of which at least a distal portion is resiliently deformable.

In various embodiments, the tension member passer body comprises a distal tip configured to penetrate tissue of the organ.

In various embodiments, the apparatus further comprising a securing member coupled to the resiliently deformable distal portion of the tension member passer body.

In various embodiments, the resiliently deformable distal portion of the tension member passer body is pre-curved, wherein the securing member comprises a wire attached to the distal portion of the tension member passer body at two distinct points, and wherein the tension member passer body and the securing member cooperate to form a loop when the deformable distal portion is positioned at an exterior of the needle.

In various embodiments, the tension member passer further comprises a distal tip configured to penetrate tissue of the organ.

In various embodiments, the tension member passer is sufficiently stiff to form a tract through the organ when advanced distally through the needle.

In various embodiments, the tension member passer comprises a resiliently deformable loop that is configured to automatically open to an expanded state when the loop is advanced distally past the needle tip and is configured to collapse to a contracted state when the loop is retracted proximally into the needle tip.

In various embodiments, the needle is configured to be advanced distally relative to the tube to a position beneath the surface of the organ, and wherein the tension member passer is configured to be advanced distally relative to the needle by an amount sufficient to penetrate the surface of the organ and thereby extend the pathway back through the surface of the organ at the exit point from the organ.

In various embodiments, the passer comprises a distal tip configured to penetrate tissue of the organ.

In various embodiments, the needle is configured to be advanced distally relative to the tube by an amount sufficient to penetrate the surface of the organ and thereby extend the pathway back through the surface of the organ at the exit point from the organ.

In various embodiments, the tube is configured to be positioned within the organ at a depth beneath the surface of the organ that is greater than a maximum depth of the tissue mass beneath the surface of the organ.

In various embodiments, the tension member passer is configured to pull the tension member through the exit opening, the passage, and the entry opening prior to retraction of the needle into the tube.

In various embodiments, the tension member passer is configured to pull the tension member through the exit opening, the passage, and the entry opening after retraction of the needle into the tube.

In various embodiments, the tension member passer is configured to pull the tension member through the exit opening, the passage, and the entry opening after retraction of the tube out of the organ.

In various embodiments, the needle comprises an opening at a distal tip of the needle.

In various embodiments, the curved path extends exclusively around an exterior of the tissue mass.

In various embodiments, the curved path extends through a portion of the tissue mass.

In various embodiments, the entry point and the exit point are spaced from each other.

In various embodiments, a distance between the entry point and the exit point is no greater than 2 times a diameter of the tissue mass.

In various embodiments, a distance between the entry point and the exit point is no greater than the diameter of the tissue mass.

In various embodiments, a distance between the entry point and the exit point is smaller than a diameter of the tissue mass.

In various embodiments, the distal portion of the needle comprises a first region defining a first radius of curvature and a second region defining a second radius of curvature, and wherein the first radius of curvature is greater than the second radius of curvature.

In various embodiments, the first radius of curvature is greater than the second radius of curvature by a factor of no less than 3.

In various embodiments, the first radius of curvature is greater than the second radius of curvature by a factor of no less than 4.

In various embodiments, the first radius of curvature is greater than the second radius of curvature by a factor of no less than 5.

In various embodiments, the first radius of curvature is greater than the second radius of curvature by a factor of no less than 6.

In various embodiments, the first radius of curvature is greater than the second radius of curvature by a factor of no less than 7.

In various embodiments, a length of the first region is greater than a length of the second region by a factor of no less than 10.

In various embodiments, a length of the first region is greater than a length of the second region by a factor of no less than 15.

In various embodiments, a length of the first region is greater than a length of the second region by a factor of no less than 20.

In various embodiments, a length of the first region is greater than a length of the second region by a factor of no less than 25.

In various embodiments, a length of the first region is greater than a length of the second region by a factor of no less than 30.

In various embodiments, the first region and the second region are adjacent to one another, and wherein the first region is proximal to the second region.

In various embodiments, the needle tip comprises at least a portion of the second region.

In various embodiments, at least a portion of the needle tip is positioned distally relative to the second region.

In various embodiments, a radially outer surface of the second region is configured to push against tissue as the needle is advanced through the organ in a manner that encourages the needle to form a tighter curved path than would be achieved if the second region were to have the same radius of curvature as the first region.

In various embodiments, the needle tip comprises a primary bevel at which an opening of the needle lumen is positioned, and wherein the second region orients the bevel to be within 10 degrees of a direction of travel of the needle tip as the needle as advanced through the organ to thereby reduce an amount of tissue coring at the opening than would be experienced if the second region were to have the same radius of curvature as the first region.

In various embodiments, the second region orients the bevel to be within 5 degrees of the direction of travel of the needle tip.

In various embodiments, the second region orients the bevel to be colinear with or parallel to the direction of travel of the needle tip.

In various embodiments, the apparatus further comprising a gripping device configured to grip the organ to stabilize the organ as the tube is advanced through the surface of the organ.

In various embodiments, the gripping device comprises a pair of tenaculum arms configured to hold a portion of the organ.

In various embodiments, the apparatus further comprising an elongated member coupled with both the gripping device and the tube, wherein the gripping device is fixedly secured to the elongated member and wherein the tube is configured to translate relative to the elongated member.

In various embodiments, the elongated member defines a lumen, and wherein the tube is configured to pass through the lumen of the elongated member.

In various embodiments, the gripping device comprises an actuator configured to remotely manipulate the gripping device.

In various embodiments, the gripping device comprises a pair of opposable arms, and wherein the actuator is configured to approximate the opposable arms toward each other.

In various embodiments, the actuator comprises a lock configured to maintain the opposable arms in a fixed orientation relative to each other when locked.

In various embodiments, the actuator comprises a lock configured to maintain the gripping device in a gripping orientation relative to the organ when locked.

In various embodiments, the tube is translatable relative to the gripping device.

In various embodiments, the apparatus further comprising a console fixedly coupled to the tube and the gripping device, wherein the console comprises a first actuator configured to control movement of the gripping device when the console is held stationary, and wherein the console comprises a second actuator configured to control translation of the tube relative to the gripping device when the console is held stationary.

### Example 4

A surgical needle can comprise: an elastic needle body ending with a sharp needle tip, the needle body includes a first segment having a first centerline, and a second segment having a second centerline, the second segment adjoins with a proximal end thereof to a distal end of the first segment and with a distal end thereof to a proximal end of a tip segment; wherein, when the needle body is in an unstressed relaxed form, the first centerline has a first radius of curvature and the second centerline has a second radius of curvature smaller than the first radius of curvature.

In various embodiments, a ratio between the second radius of curvature and the first radius of curvature is within a range of 1/10 to ⅓, optionally between about 1/7 and about ⅕.

In various embodiments, when the needle body is in the unstressed relaxed form, the first segment forms a first outer arc subtending a first subtended angle and/or the second segment forms a second outer arc subtending a second subtended angle, wherein the second subtended angle is smaller than the first subtended angle.

In various embodiments, the first subtended angle is within a range of 200° to 300°, optionally between 240° and 260°, and/or the second subtended angle is within a range of 10° to 80°, optionally between 30° and 60°.

In various embodiments, the tip segment has a tridimensional beveled shape formed by adjoined curved outer surface and flat inner surface intersecting at the needle tip, wherein outline of the outer surface extends from a point on second segment distal end located radially outwardly to the second centerline and outline of the inner surface extends from a point provided between second segment proximal end and second segment distal end located radially inwardly to the second centerline, relative to the second radius of curvature.

In various embodiments, the needle body encloses a needle lumen with a needle opening in proximity to the needle tip, wherein the inner surface surrounds the needle opening.

In various embodiments, an intersection angle defined by intersection of the outer surface and the inner surface at the needle tip is within a range of 10° to 30°, optionally about 20°.

In various embodiments, the outline of the outer surface is straight.

In various embodiments, the outline of the outer surface is shorter than the outline of the inner surface, optionally about half in length thereof.

In various embodiments, the inner surface is tilted radially inwardly relative to a forward tangent of the first segment distal end.

In various embodiments, the first radius of curvature is within a range of 15 mm to 45 mm when the needle body is in the unstressed relaxed state.

In various embodiments, the needle body is configured with elastic resistance to straightening within a range of 2 N to 20 N.

### Example 5

A surgical apparatus can comprise: a rigid outer tube comprising a sharp outer tube tip and an outer tube lumen with an outer tube opening in proximity to the outer tube tip; and an inner needle configured as the surgical needle according to Example 4; wherein the inner needle body is configured to pass straightened through the outer tube lumen and to partially protrude via the outer tube opening, such that a protruding portion of the inner needle body is allowed to voluntarily flex to a curved form, whereby the inner needle tip is configured to follow a first curved path having a radius of curvature similar to the second radius of curvature, when the protruding portion excludes the first segment distal end, and to shift into following a second curved path having a radius of curvature similar to or smaller than the first radius of curvature, when the protruding portion includes the first segment distal end.

### Example 6

A method can comprise: penetrating a soft tissue with a sharp outer tube tip of a rigid outer tube enclosing an outer tube lumen with an outer tube opening in proximity to the outer tube tip; passing an inner needle through the outer tube lumen, the inner needle comprising an elastic inner needle body ending with a sharp inner needle tip, the inner needle body includes a first segment having a first centerline, and a second segment having a second centerline, the second segment adjoins with a proximal end thereof to a distal end of the first segment and with a distal end thereof to a proximal end of a tip segment, wherein, when the needle body is in an unstressed relaxed form, the first centerline has a first radius of curvature and the second centerline has a second radius of curvature smaller than the first radius of curvature; initially advancing the inner needle tip in the soft tissue via the outer tube opening while increasing length of a protruding portion of the inner needle body until reaching a first penetration depth such that the protruding portion excludes the first segment distal end, thereby allowing the inner needle tip to follow a first curved path having a radius of curvature similar to the second radius of curvature; and further advancing the inner needle tip in the soft tissue via the outer tube opening while increasing length of the protruding portion until reaching a second penetration depth such that the protruding portion includes the first segment distal end, thereby allowing the inner needle tip to follow a second curved path having a radius of curvature similar to or smaller than the first radius of curvature and greater than the second radius of curvature.

In various embodiments, the further advancing the inner needle includes transferring a second force vector through the first segment to the second segment and the tip segment, and from both the second segment and the tip segment to tissue surrounding thereto.

In various embodiments, a tangential force component of the second force vector tangent to the second curved path is smaller than a normal force component of the second force vector perpendicular to the second curved path.

In various embodiments, the initially advancing the inner needle includes transferring a first force vector through the first segment to the second segment and the tip segment, applied from the tip segment to tissue surrounding thereto.

In various embodiments, a tangential force component of the first force vector tangent to the first curved path is greater than a normal force component of the first force vector perpendicular to the first curved path.

In various embodiments, a normal force component of the second force vector is greater than a normal force component of the first force vector.

In various embodiments, the tip segment has a tridimensional beveled shape formed by adjoined curved outer surface and flat inner surface intersecting at the needle tip, wherein outline of the outer surface extends from a point on second segment distal end located radially outwardly to the second centerline and outline of the inner surface extends from a point provided between second segment proximal end and second segment distal end located radially inwardly to the second centerline, relative to the second radius of curvature, the needle body encloses an inner needle lumen with an inner needle opening in proximity to the needle tip, the inner needle opening is surrounded with the inner surface; wherein the further advancing the inner needle includes forming a surgical passage within the organ by forcing the outer surface against the tissue surrounding the second segment and the tip segment in direction of the second force vector, thereby preventing coring of penetrated tissue via the inner needle opening.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosure.

Throughout this disclosure, a numerical value of a parameter, feature, characteristic, object, or dimension, may be stated or described in terms of a numerical range format. Such a numerical range format, as used herein, illustrates implementation of some exemplary embodiments, and does not inflexibly limit the scope of the exemplary embodiments. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

All publications, patents, and or/and patent applications, cited or referred to in this disclosure are herein incorporated in their entirety by reference into the specification, to the same extent as if each individual publication, patent, or/and patent application, was specifically and individually indicated to be incorporated herein by reference. In addition, citation or identification of any reference in this specification shall not be construed or understood as an admission that such reference represents or corresponds to prior art of the present invention. To the extent that section headings are used, they should not be construed as necessarily limiting.

1-36. (canceled)

37. A surgical needle, comprising:
an elastic needle body ending with a sharp needle tip, the needle body includes a first segment, a second segment and a tip segment, the second segment extending distally from the first segment to the tip segment;
wherein, when the needle body is in an elastically relaxed state, a first centerline of the first segment has a first radius of curvature and a second centerline of the second segment has a second radius of curvature smaller than the first radius of curvature.

38. The surgical needle according to claim 37, wherein the first radius of curvature and/or the second radius of curvature is constant, and/or a ratio between the second radius of curvature and the first radius of curvature is within a range of 1/10 to ⅓.

39. The surgical needle according to claim 37, wherein when the needle body is in the elastically relaxed state, the first segment forms a first outer arc subtending a first subtended angle and/or the second segment forms a second outer arc subtending a second subtended angle, wherein the second subtended angle is smaller than the first subtended angle.

40. The surgical needle according to claim 39, wherein the first subtended angle is within a range of 200° to 300°, and/or the second subtended angle is within a range of 10° to 80°.

41. The surgical needle according to claim 37, wherein the tip segment has a tridimensional beveled shape formed by a curved outer surface and a flat inner surface intersecting at the needle tip, wherein an outline of the outer surface extends from a point on a distal end of the second segment located radially outwardly to the second centerline, and wherein an outline of the inner surface extends from a point provided along the second segment located radially inwardly to the second centerline, relative to the second radius of curvature.

42. The surgical needle according to claim 41, wherein the needle body encloses a needle lumen with a needle opening in proximity to the needle tip, wherein the inner surface surrounds the needle opening.

43. The surgical needle according to claim 41, wherein an intersection angle defined by intersection of the outer surface and the inner surface at the needle tip is within a range of 10° to 30°.

44. The surgical needle according to claim 41, wherein the outline of the outer surface is straight.

45. The surgical needle according to claim 41, wherein the outline of the outer surface is shorter than the outline of the inner surface.

46. The surgical needle according to claim 41, wherein the inner surface is tilted radially inwardly relative to a forward tangent of a distal end of the first segment.

47. The surgical needle according to claim 37, wherein the first radius of curvature is within a range of 15 mm to 45 mm when the needle body is in the elastically relaxed state.

48. The surgical needle according to claim 37, wherein the needle body is configured with elastic resistance to straightening within a range of 2 N to 20 N.

49. A surgical apparatus, comprising:
a rigid outer tube comprising a sharp outer tube tip and an outer tube lumen with an outer tube opening in proximity to the outer tube tip; and
an inner needle comprising a body ending with a sharp needle tip, wherein the inner needle body includes a first segment, a second segment and a tip segment, the second segment extending distally from the first segment to the tip segment;
wherein, when the needle body is in an elastically relaxed state, a first centerline of the first segment has a first radius of curvature and a second centerline of the second segment has a second radius of curvature smaller than the first radius of curvature;
wherein the inner needle body is configured to pass straightened through the outer tube lumen and to partially protrude via the outer tube opening, such that a protruding portion of the inner needle body is allowed to voluntarily flex to a curved form, whereby the inner needle tip is configured to follow a first curved path having a radius of curvature similar to the second radius of curvature, when the protruding portion excludes the first segment distal end, and to shift into following a second curved path having a radius of curvature similar to or smaller than the first radius of curvature, when the protruding portion includes the first segment distal end.

50. An apparatus according to claim 49, further comprising a forceps head fixedly positioned distally to the console and activatable via the console, configured for selective grasping of the organ adjacent to the entry point and/or the exit point, for holding the grasped organ at a fixed distance relative to the console.

51. A method, comprising:
penetrating a soft tissue with a sharp outer tube tip of a rigid outer tube enclosing an outer tube lumen with an outer tube opening in proximity to the outer tube tip;
passing an inner needle through the outer tube lumen, the inner needle comprising an elastic inner needle body ending with a sharp inner needle tip, the inner needle body includes a first segment having a first centerline, and a second segment having a second centerline, the second segment extends between the first segment and a tip segment, wherein, when the needle body is in an elastically relaxed state, the first centerline has a first radius of curvature and the second centerline has a second radius of curvature smaller than the first radius of curvature;
initially advancing the inner needle tip in the soft tissue via the outer tube opening while increasing length of a protruding portion of the inner needle body until reaching a first penetration depth such that the protruding portion excludes a distal end of the first segment, thereby allowing the inner needle tip to follow a first curved path having a radius of curvature similar to the second radius of curvature; and
further advancing the inner needle tip in the soft tissue via the outer tube opening while increasing length of the protruding portion until reaching a second penetration depth such that the protruding portion includes the first segment distal end, thereby allowing the inner needle tip to follow a second curved path having a radius of curvature similar to or smaller than the first radius of curvature and greater than the second radius of curvature.

52. The method according to claim 51, wherein the further advancing the inner needle includes transferring a second force vector through the first segment to the second segment and the tip segment, and from both the second segment and the tip segment to tissue surrounding thereto.

53. The method according to claim 52, wherein a tangential force component of the second force vector tangent to the second curved path is smaller than a normal force component of the second force vector perpendicular to the second curved path.

54. The method according to claim 52, wherein the initially advancing the inner needle includes transferring a first force vector through the first segment to the second segment and the tip segment, applied from the tip segment to tissue surrounding thereto.

55. The method according to claim 54, wherein a tangential force component of the first force vector tangent to the first curved path is greater than a normal force component of the first force vector perpendicular to the first curved path; and/or wherein a normal force component of the second force vector is greater than a normal force component of the first force vector.

56. The method according to claim 52, wherein the tip segment has a tridimensional beveled shape formed by adjoined curved outer surface and flat inner surface intersecting at the needle tip,
wherein an outline of the outer surface extends from a point on a distal end of the second segment located radially outwardly to the second centerline and wherein an outline of the inner surface extends from a point provided along second segment located radially inwardly to the second centerline, relative to the second radius of curvature,
the needle body encloses an inner needle lumen with an inner needle opening in proximity to the needle tip, the inner needle opening is surrounded with the inner surface;
wherein the further advancing the inner needle includes forming a surgical passage within the organ by forcing the outer surface against the tissue surrounding the second segment and the tip segment in direction of the second force vector, thereby preventing coring of penetrated tissue via the inner needle opening.
